(19) United States
(12) Patent Application Publication
(10) Pub. No.: US 2016/0039874 A1
(43) Pub. Date: Feb. 11, 2016

(54) REGULATING BACILLUS ANTHRACIS LETHAL FACTOR ACTIVITY VIA AN ACTIVATING EPITOPE REGION

(71) Applicant: The United States of America, as Represented by th Secretart, Department of Health and Human Service, Rockville, MD (US)

(72) Inventors: Jason Marc Goldstein, Decatur, GA (US); Conrad P. Quinn, Lilburn, GA (US); Dennis A. Bagarozzi, Lilburn, GA (US); Anne E. Boyer, Atlanta, GA (US)

(21) Appl. No.: 14/427,329

(22) PCT Filed: Sep. 11, 2013
(86) PCT No.: PCT/US2013/059179

§ 371 Date: Mar. 11, 2015

Related U.S. Application Data
(60) Provisional application No. 61/699,738, filed on Sep. 11, 2012.

(51) Int. Cl. C07K 7/08; C07K 7/06; A61K 39/07; C12N 9/54

(52) U.S. Cl. CPC C07K 7/08; C12N 9/54; C07K 7/06; A61K 39/07; C12Y 304/24083

(57) ABSTRACT

Provided are peptides capable of reducing the ability of antibodies to stimulate the activity of B. anthracis lethal factor. These peptides may be used to reduce activity of LF in a sample containing an LF activity enhancing antibody, to prevent the possibility of LF enhanced activity in a sample or subject, or as a therapeutic to reduce LF activity in a subject that may-have one or more antibodies that enhance LF activity. Also, provided are immunogens that may be used in a vaccine to confer protection to a subject. An immunogen does not include a sequence including the wild-type amino acids present in LF residues 677-680, or mutations of amino acids 677-680 that will also generate antibodies that enhance LF activity.

### CROSS REFERENCE TO RELATED APPLICATIONS

This application depends from and claims priority to U.S. Provisional Application No. 61/699,738 filed Sep. 11, 2012, the entire contents of which are incorporated herein by reference.

### GOVERNMENT INTEREST

The invention described herein may be manufactured, used, and licensed by or for the United States Government.

### FIELD OF THE INVENTION

The invention relates generally to disease diagnostics and therapeutics, and in particular to methods for detecting infection of anthrax or treating or preventing anthrax infection in a patient and screening anthrax therapeutics.

### BACKGROUND OF THE INVENTION

Anthrax is caused by infection with Bacillus anthracis, a spore-forming, rod-shaped bacterium. The dormant spore-form is highly resistant to extreme conditions, high temperatures, and a variety of chemical treatments. The spores gain entry either through an open wound, causing cutaneous disease, or by ingestion, causing gastrointestinal disease or are inhaled causing inhalation anthrax. All three forms can progress to a systemic infection leading to shock, respiratory failure, and death. (Mock, M. and Mignot, T. (2003), Cell Microbiol., 5(1):15-23). The stability of the spores and their infectious capacity make them a convenient bioterrorist weapon.

The two known toxins of B. anthracis are binary combinations of protective antigen (PA), named for its ability to induce protective immunity against anthrax, with either edema factor (EF) or lethal factor (LF). PA is the cell binding component of both toxins and is responsible for bringing the catalytic EF or LF into the host cells. EF is an adenylate cyclase that converts ATP to cyclic AMP and causes edema (Brossier, F. and Mock, M. (2001), Toxicon. 39(11):1747-55). The combination of PA-EF forms edema toxin (ETx) which causes edema when injected locally. LF is a zinc-dependent endoprotease known to target the amino-terminus of the mitogen-activated protein kinase kinase (MAPKK) family of response regulators (Id.). The cleavage of these proteins disrupts a signaling pathway and leads to cytokine dysregulation and immune dysfunction. LF combined with PA forms lethal toxin (LTx), which is lethal when injected on its own. It is also known that there are fatal anthrax cases where administration of antibiotics and clearance of bacteria have failed to rescue the patient. This indicates that there may be a “point of no return” level of LTx in the blood that may predict the outcome of infection. Clearly, LTx and its components are important targets for diagnostics and quantification.

Assays for EF activity such as competitive enzyme assays (Duriez, E, et al., Anal. Chem., 2009; 81:5935-5941) or radiometric assays (Gottle, M, et al., Biochemistry, 2010; 49:5494-503), are impractical for high-throughput screening of compound collections and rapid diagnosis of host infection. Methods for rapid screening of patients in a hospital setting or identification of potent and selective EF inhibitors requires an assay that is less labor intensive, has faster turnaround, and is effective at low levels of enzyme.

Development of targeted therapies following anthrax infection is essential to managing a patient population. As such, there exists a need for compositions and methods that can be used as aids in a screening assay for identification and development of inhibitors of anthrax toxin activity or for identifying active LF such as in the circulation of a subject.

### SUMMARY OF THE INVENTION

The following summary of the invention is provided to facilitate an understanding of some of the innovative features unique to the present invention and is not intended to be a full description. A full appreciation of the various aspects of the invention can be gained by taking the entire specification, claims, drawings, and abstract as a whole.

The invention provides a peptide sequence that represents an epitope sequence in B. anthracis lethal factor that when recognized by antibodies such as AVR1674 and AVR1675 allows these LF stimulatory antibodies to function. It is one object of the invention to provide a peptide including a sequence that may be used as a competitive inhibitor of antibody enhanced LF activity. A peptide is or includes a peptide sequence included in the consensus sequence (T/S)-X₁-(K/R)-(D/E), optionally, (T/S)-X₁-(K/R)-(D/E)-X₂-X₃. Incubation of peptides having the consensus sequence will reduce antibody enhanced LF activity. It has been demonstrated that peptides having the consensus sequence are specifically recognized by antibodies that will enhance LF activity.

A peptide may be isolated partially or totally. In some embodiments, X₁ is phenylalanine, alanine, or histidine. In some embodiments, the amino acid at position 4 is glutamate. In some embodiments, X₃ is isoleucine or alanine. It is appreciated that these and other embodiments, may be combined in any way. Optionally, the peptide is or includes the sequence of TFKDEI (SEQ ID NO: 4).

Also provided is a host cell that includes a vector encoding a peptide that is or includes the sequence of (T/S)-X₁-(K/R)-(D/E)-X₂-X₃ or TFKDEI (SEQ ID NO: 4). As such, it is another object of the invention to provide one or more sources to produce one or more peptides having a sequence that falls under the consensus sequence provided.

It is another object of the invention to provide a process of reducing enhancement of B. anthracis lethal factor activity including combining B. anthracis lethal factor with a peptide having a sequence included in the sequence (T/S)-X₁-(K/R)-(D/E)-X₂-X₃. The combination of LF with a peptide is optionally in an aqueous medium. The peptide is recognized is recognized by an antibody that enhances LF activity such that the peptide acts as a competitive inhibitor to antibody induces LF activity enhancement. In the process, any peptide having the sequence (T/S)-X₁-(K/R)-(D/E) or (T/S)-X₁-(K/R)-(D/E)-X₂-X₃ may be used. Optionally, the medium also includes an antibody that recognizes an epitope comprising the sequence of THQDEIFEQK (SEQ ID NO: 41), or portion thereof. In a process, an antibody is optionally AVT1674. Optionally, a peptide used in a process is or includes the sequence TFKDEI (SEQ ID NO: 4).

Also provided are processes of treating a human or non-human subject including administering the subject a peptide that is or includes a sequence that is included in the consensus sequence (T/S)-X₁-(K/R)-(D/E), optionally, (T/S)-X₁-(K/R)-(D/E)-X₂-X₃. A peptide may be isolated partially or totally. In some embodiments, X₁ is phenylalanine, alanine, or histidine. In some embodiments, the amino acid at position 4 is glutamate. In some embodiments, X₃ is isoleucine or alanine. It is appreciated that these and other embodiments, may be combined in any way. Optionally, the peptide is or includes the sequence of TFKDEI (SEQ ID NO: 4).

It is another object of the invention to provide a therapeutic composition that includes a peptide that is or includes a sequence that is included in the consensus sequence (T/S)-X₁-(K/R)-(D/E), optionally (T/S)-X₁-(K/R)-(D/E)-X₂-X₃, and a pharmaceutical carrier.

Also provided are immunogens that can be administered to a subject to induce immunity from or protection against complications from infection by B. anthracis. An immunogen may include any sequence of LF but is absent a region that may lead to the production of antibodies with a stimulatory effect on LF activity. An immunogen includes a portion of B. anthracis lethal factor peptide, wherein said peptide includes amino acid sequences from the two regions that flank residues 677-680, 677-686, or any portion or the whole of residues 667-688 representing the L2 region, where the peptide excludes the sequence of residues 677-680 of SEQ ID NO: 2 or does not have a sequence falling under the consensus sequence (T/S)-X₁-(K/R)-(D/E). The immunogen optionally includes a portion of the lethal factor domain IV where the domain IV includes a deletion relative to SEQ ID NO: 2, wherein at least one amino acid of residues 677-680 of SEQ ID NO: 2, optionally, the amino acid at P1, P3, P4, or combinations thereof is deleted, or substituted to fall outside the consensus sequence (T/S)-X₁-(K/R)-(D/E). An immunogen optionally does not include a full length LF region. An immunogen optionally does not include any portion of the L2 region. In some embodiments, an immunogen includes a portion of the lethal factor domain IV, the domain including an amino acid substitution of one or more of residues 677-680 of SEQ ID NO: 2 wherein P1 is neither T nor S, P3 is neither K nor R, P4 is neither D nor E, or combinations thereof. In some embodiments, an immunogen has a substitution of the entire L2 region of LF with a peptide of 2 or more amino acids, optionally the amino acids are alanine, glycine, or combinations thereof.

### BRIEF DESCRIPTION OF THE DRAWINGS

FIG. 1 is a summary of ELISA results from peptides having sequences identified in phage display analyses illustrating high affinity binding (defined as an O.D. of 0.5 or greater) for many sequences;

FIG. 2 illustrates inhibition of antibody binding to LF by the phage displaying peptide AVR-1674 C8;

FIG. 3A illustrates enhancement of LF activity by antibodies AVR1674 and AVR1675 at 2 hours of incubation;

FIG. 3B illustrates enhancement of LF activity by antibodies AVR1674 and AVR1675 at 16 hours of incubation;

FIG. 3C illustrates enhancement of LF activity by antibodies AVR1674 and AVR1675 at 5 hours of incubation;

FIG. 4A illustrates a concentration dependent activation of LF by antibody AVR1674; and

FIG. 4B illustrates a concentration dependent activation of LF by antibody AVR1675.

### DETAILED DESCRIPTION OF EMBODIMENTS OF THE INVENTION

The following description of particular embodiment(s) is merely exemplary in nature and is in no way intended to limit the scope of the invention, its application, or uses, which may, of course, vary. The invention is described with relation to the non-limiting definitions and terminology included herein. These definitions and terminology are not designed to function as a limitation on the scope or practice of the invention but are presented for illustrative and descriptive purposes only. While the process is described as an order of individual steps or using specific materials, it is appreciated that described steps or materials may be interchangeable such that the description of the invention includes multiple parts or steps arranged in many ways as is readily appreciated by one of skill in the art.

A recent mass spectrometry assay was developed using antibodies that specifically target Bacillus anthracis lethal factor (LF) without neutralizing the levels of LF in an organism or reducing the activity of LF in an assay system. Boyer et al., Anal. Chem., 2007; 79 (22):8463-8470. The inventors discovered that two monoclonal antibodies useful in this assay, AVR1674 and AVR1675, enhance LF activity. The mechanism of LF activity enhancement or the epitopes of these antibodies were previously unknown. The epitope is presented herein for the first time allowing for the identification and development of compositions useful in diagnostics or as therapeutics. As such, the invention has utility to aid in the identification and development of therapeutics for the treatment or prevention of Bacillus anthracis infection, or as a composition useful for the screening or other detection of Bacillus anthracis lethal factor (LF) in a sample.

The inventors discovered that antibody AVR1674 and AVR1675 recognize a particular epitope in LF leading to stimulation of LF activity indicating that modulation of this region by antibody interactions may detract from the effectiveness of vaccines or naturally derived immune responses to the presence of LF in the circulation. The epitope was determined by significant effort to include the sequence THQDEIYEQV (SEQ ID NO. 1), which represents residues 677-686 of the immature full length LF protein as found at Swiss-Prot Accession No: P15917 and SEQ ID NO: 2, and in particular the first six residues of SEQ ID NO: 1.

| (SEQ ID NO: 2) | |
|---|---|
| 1 | mnikkefikv ismsclvtai tlsgpvfipl vqgagghgdv gmhvkekekn kdenkrkdee |
| 61 | rnktqeehlk eimkhivkie vkgeeavkke aaekllekvp sdvlemykai ggkiyivdgd |
| 121 | itkhisleal sedkkkikdi ygkdallheh yvyakegyep vlviqssedy ventekalnv |
| 181 | yyeigkilsr dilskinqpy qkfldvlnti knasdsdgqd llftnqlkeh ptdfsvefle |
| 241 | qnsnevqevf akafayyiep qhrdvlqlya peafnymdkf neqeinlsle elkdqrmlar |
| 301 | yekwekikqh yqhwsdslse egrgllkklq ipiepkkddi ihstsqeeke llkriqidss |
| 361 | dflsteekef lkklqidird slseeekell nriqvdssnp lsekekeflk klkldiqpyd |
| 421 | inqrlqdtgg lidspsinld vrkqykrdiq nidallhqsi gstlynkiyl yenmninnlt |
| 481 | atlgadlvds tdntkinrgi fnefkknfky sissnymivd inerpaldne rlkwriqlsp |
| 541 | dtragyleng klilqrnigl eikdvqiikq sekeyirida kvvpkskidt kiqeaqlnin |
| 601 | qewnkalglp kytklitfnv hnryasnive saylilnewk nniqsdlikk vtnylvdgng |
| 661 | rfvftditlp niaeqythqd eiyeqvhskg lyvpesrsil lhgpskgvel rndsegfihe |
| 721 | fghavddyag ylldknqsdl vtnskkfidi fkeegsnlts ygrtneaeff aeafrlmhst |
| 781 | dhaerikvqk napktfqfin dqikfiins |

For anthrax toxin LF the mature secreted protein (776 aa; Mᵣ 90237) is preceded by a 33-aa signal peptide (Bragg and Robertson, Gene. 1989 Sep. 1; 81(1):45-54), which is included in the above sequence for immature LF. Mature LF, therefore, has this signal sequence removed and the numbering adjusted accordingly.

A peptide for use in reducing antibody enhanced LTx or LF activity includes the amino acid consensus sequence (T/S)-X₁-(K/R)-(D/E)-X₂-X₃ wherein X₁ is any amino acid, optionally phenylalanine, alanine, or histidine. In some embodiments, a peptide has a sequence (T/S)-X₁-(K/R)-(D/E)-X₂-X₃ where: X₁ is any amino acid, optionally phenylalanine, alanine, or histidine; X₂ is any amino acid, optionally aspartic acid or glutamic acid; and X₃ is any non-polar or hydrophobic amino acid, optionally isoleucine or alanine. The amino acid sequence (T/S)-X₁-(K/R)-(D/E) is optionally used alone or as part of a larger peptide that encompasses an epitope for a stimulatory antibody. Optionally, the sequence (T/S)-X₁-(K/R)-(D/E) is attached to a linker peptide. A linker peptide is optionally attached to the C-terminal. In some embodiments, a linker peptide has the sequence GGGSK (SEQ ID NO: 3). A peptide has from 4 to 100 amino acids, or any value or range therebetween. A peptide optionally has 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, or 100 amino acids as long as the sequence (T/S)-X₁-(K/R)-(D/E), optionally (T/S)-X₁-(K/R)-(D/E)-X₂-X₃, is present in the peptide. In some embodiments, a peptide includes the sequence TFKDEI (SEQ ID NO: 4) or variants thereof. In some embodiments, a variant is a peptide that includes the sequence (T/S)-X₁-(K/R)-(D/E)-X₂-X₃ wherein: the amino acids at position P1, P3 and P4 are essential and limited as described, X₁ is preferably F, A, or H with preference in that order; the amino acid at X₂ is preferably E; or the amino acid at position X₃ is preferably A. Optionally, a peptide does not have the wild type sequence THQDEIYEQV (SEQ ID NO. 1) or a wild type fragment thereof absent a non-wild type sequence amino acid within or attached to the peptide, or the presence of an other non-wild type element(s).

A peptide as defined herein includes variants that are conservative substitutions of desirable amino acids. Illustratively, a variant is a conservative substitution of residues in (T/S)-X₁-(K/R)-(D/E) at P2, or (T/S)-X₁-(K/R)-(D/E)-X₂-X₃ at P2, P5, or P6, that may or may not be the substitutions listed above. A conservative amino acid substitution is recognized by one of skill in the art.

Also provided is an immunogen for use as a vaccine for prophylaxis or treatment of anthrax infection. An immunogen is a peptide or protein corresponding one or more regions of LF according to SEQ ID NO: 2, the mature LF sequence, or a variant thereof, that does not include a L2 region in domain IV including the sequence (TIS)-X₁-(K/R)-(D/E). An immunogen, therefore, capitalizes on the recognition that the sequence (T/S)-X₁-(K/R)-(D/E) is a minimal epitope conserved region for antibody enhancement of LF activity. By eliminating or altering this sequence of LF, the molecule or portion thereof can be used as a superior vaccine to protect against the effects of anthrax infection by reducing or eliminating the likelihood of natural development of LF activity enhancing antibodies. This region of LF that is to be excluded or altered in an immunogen maps to the L2 region of the catalytic domain IV of LF and forms a solvent exposed loop. Immunization of a subject with an inventive immunogen or nucleic acid sequence encoding this immunogen will elicit an immune response in the subject leading to toxin neutralization and reduction or elimination of undesirable affects of Bacillus anthracis infection while reducing or eliminating the risk of developing a LF activity enhancing antibody.

An immunogen as defined herein includes one or more amino acids of domain IV in LF on each side of the sequence of residues 677-680 in the L2 loop as found in SEQ ID NO: 2, or conservative mutations thereof, but need not be the residues immediately adjacent to residues 677-680. As such, an immunogen includes both N- and C-terminal amino acids representative of LF that flank residues 677-680, the L2 sequence, or any portion of the L2 sequence excluding the residues 677-680, but need not be immediately adjacent thereto. It is appreciated that these residues may, but are not required to in some embodiments, possess the residues of SEQ ID NO: 2 immediately adjacent to the L2 loop, but will include 3 or more amino acids that are found on each side of any deleted portion of the L2 loop in domain TV of LF, or conservative mutations thereof. As such, an immunogen includes at minimum 6 amino acids representing at least portions of domain IV of LF that exclude one or more amino acids of the sequence (T/S)-X₁-(K/R)-(D/E). Specific examples of an immunogen are LF domain IV deletions of SEQ ID NO: 2 with a deletion of residues 677-680, 677-686, or any portion or the whole of residues 667-688 representing the L2 region that includes residues 677-680. Illustrative examples of a deletion mutation include deletion of one or more residues of the sequence (T/S)-X₁-(K/R)-(D/E) representing residues 677-680 of SEQ ID NO: 2, optionally, the amino acid at P1, P3, P4, or combinations thereof are deleted.

An immunogen is optionally a portion of LF that includes amino acid sequences from the two regions that flank residues 677-680, 677-686, or any portion or the whole of residues 667-688 representing the L2 region that excludes residues 677-680. Illustratively, an immunogen includes all or a portion of LF domain I, II, III, or IV, and also includes amino acid sequences from the two regions that flank residues 677-680, 677-686, or any portion or the whole of residues 667-688 representing the L2 region that excludes residues 677-680. Illustrative examples of an immunogen and methods of immunogen preparation are described in Quinn et al., J. Biol. Chem., 1991; 266:20124-20130. The structure of LF and location of the L2 region in domain IV and its proximity to the LF active site are found in Dalkas, et al., Protein Science, 2009; 18: 1774-1785.

Optionally, an immunogen includes the L2 region with a substitution within the sequence (T/S)-X₁-(K/R)-(D/E) where P1 is neither T nor S, P3 is neither K nor R, P4 is neither D nor E, or combinations thereof. One or more essential residues in the consensus sequence (T/S)-X₁-(K/R)-(D/E) is substituted by an alanine, glycine, or other amino acid resulting in a sequence that is not recognized by a LF function enhancing antibody such as AVR1674 and AVR1675.

In some aspects, vaccines are provided alone or in combination with a carrier, adjuvant, or other component that, when administered to a subject such as a human or other animal, induces the production of antibodies that recognize LF without recognition of residues 677-680 of SEQ ID NO: 2. Immunization is achieved by administering to a subject an immunogenic amount of one or more of the immunogens in a pharmaceutically acceptable carrier. An immunogenic amount is any amount that will produce an immunological response, illustratively the production of antibodies, in a subject to which the immunogen is administered.

As used herein, the terms “subject” or “organism” are treated synonymously and are defined as any organism capable of hosting infection of bacteria, illustratively Bacillus anthracis. A subject illustratively includes: any mammal such as humans, non-human primates, horses, goats, cows, sheep, pigs, dogs, cats, rodents; as well as cells. In some embodiments, a subject is a human and excludes other organisms. In some embodiments, a subject is a cell and is exclusive of an organism.

A therapeutically effective amount is defined as an amount of an inventive compound that when administered to a subject, will reduce enhancement of LF activity at any measureable level. When a molecule is administered as an immunogen, an effective amount is any amount that will produce one or more immune responses in a subject, where an immune response is a response recognized in the art as expected.

The terms “biologically active peptide” and “peptide therapeutic agent,” “peptide,” “polypeptide,” “protein”, and “immunogen” are used herein and are intended to mean a natural or synthetic compound containing 4 or more amino acids. It is appreciated that a “nucleic acid immunogen” as used herein includes one or more nucleic acids. It is further appreciated that the description of elements of a peptide are also appreciated as corresponding to description of an immunogen that is not a nucleic acid immunogen. Amino acids present in a peptide include the common amino acids alanine, cysteine, aspartic acid, glutamic acid, phenylalanine, glycine, histidine, isoleucine, lysine, leucine, methionine, asparagine, proline, glutamine, arginine, serine, threonine, valine, tryptophan, and tyrosine as well as less common naturally occurring amino acids, modified amino acids or synthetic compounds, such as alpha-asparagine, 2-aminobutanoic acid or 2-aminobutyric acid, 4-aminobutyric acid, 2-aminocapric acid (2-aminodecanoic acid), 6-aminocaproic acid, alpha-glutamine, 2-aminoheptanoic acid, 6-aminohexanoic acid, alpha-aminoisobutyric acid (2-aminoalanine), 3-aminoisobutyric acid, beta-alanine, allo-hydroxylysine, allo-isoleucine, 4-amino-7-methylheptanoic acid, 4-amino-5-phenylpentanoic acid, 2-aminopimelic acid, gamma-amino-beta-hydroxybenzenepentanoic acid, 2-aminosuberic acid, 2-carboxyazetidine, beta-alanine, beta-aspartic acid, biphenylalanine, 3,6-diaminohexanoic acid, butanoic acid, cyclobutyl alanine, cyclohexylalanine, cyclohexylglycine, N5-aminocarbonylornithine, cyclopentyl alanine, cyclopropyl alanine, 3-sulfoalanine, 2,4-diaminobutanoic acid, diaminopropionic acid, 2,4-diaminobutyric acid, diphenyl alanine, N,N-dimethylglycine, diaminopimelic acid, 2,3-diaminopropanoic acid, S-ethylthiocysteine, N-ethylasparagine, N-ethylglycine, 4-aza-phenylalanine, 4-fluoro-phenylalanine, gamma-glutamic acid, gamma-carboxyglutamic acid, hydroxyacetic acid, pyroglutamic acid, homoarginine, homocysteic acid, homocysteine, homohistidine, 2-hydroxyisovaleric acid, homophenylalanine, homoleucine, homoproline, homoserine, homoserine, 2-hydroxypentanoic acid, 5-hydroxylysine, 4-hydroxyproline, 2-carboxyoctahydroindole, 3-carboxyisoquinoline, isovaline, 2-hydroxypropanoic acid (lactic acid), mercaptoacetic acid, mercaptobutanoic acid, sarcosine, 4-methyl-3-hydroxyproline, mercaptopropanoic acid, norleucine, nipecotic acid, nortyrosine, norvaline, omega-amino acid, ornithine, penicillamine (3-mercaptovaline), 2-phenylglycine, 2-carboxypiperidine, sarcosine (N-methylglycine), 2-amino-3-(4-sulfophenyl)propionic acid,1-amino-1-carboxycyclopentane, 3-thienylalanine, epsilon-N-trimethyllysine, 3-thiazolylalanine, thiazolidine 4-carboxylic acid, alpha-amino-2,4-dioxopyrimidinepropanoic acid, and 2-naphthylalanine. Accordingly, the “peptide” as used herein may include peptides having between 4 and about 1000 amino acids or having a molecular weight in the range of about 150-350,000 Daltons.

Modifications and changes can be made in the sequence of a peptide and still obtain a molecule capable of reducing enhancement of LF activity. For example, certain amino acids can be substituted for other amino acids in a sequence without appreciable loss of activity. Because it is the interactive capacity and nature of a peptide that defines that peptide's biological functional activity, certain amino acid sequence substitutions can be made in a peptide sequence and nevertheless obtain a polypeptide with like properties.

In making such changes, the hydropathic index of amino acids can be considered. The importance of the hydropathic amino acid index in conferring interactive biologic function on a polypeptide is generally understood in the art. It is known that certain amino acids can be substituted for other amino acids having a similar hydropathic index or score and still result in a polypeptide with similar biological activity. Each amino acid has been assigned a hydropathic index on the basis of its hydrophobicity and charge characteristics. Those indices are: isoleucine (+4.5); valine (+4.2); leucine (+3.8); phenylalanine (+2.8); cysteine/cysteine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is believed that the relative hydropathic character of the amino acid determines the secondary structure of the resultant polypeptide, which in turn defines the interaction of the polypeptide with other molecules, such as enzymes, substrates, receptors, antibodies, antigens, and the like. It is known in the art that an amino acid can be substituted by another amino acid having a similar hydropathic index and still obtain a functionally equivalent polypeptide. In such changes, the substitution of amino acids whose hydropathic indices are within ±2 are optional, those within ±1 are optional preferred, and those within ±0.5 are optional.

Substitution of like amino acids can also be made on the basis of hydrophilicity, particularly, where the biological functional equivalent polypeptide or peptide thereby created is intended. The following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); proline (−0.5±1); threonine (−0.4); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). It is understood that an amino acid can be substituted for another having a similar hydrophilicity value and still obtain a biologically equivalent, and in particular, an activity equivalent or superior peptide. In such changes, the substitution of amino acids whose hydrophilicity values are within ±2 is preferred, those within ±1 are particularly preferred, and those within ±0.5 are even more particularly preferred.

As outlined above, amino acid substitutions are generally based on the relative similarity of the amino acid side-chain substituents, for example, their hydrophobicity, hydrophilicity, charge, size, and the like. Such substitutions are typically considered conservative substitutions. Exemplary conservative substitutions that take various of the foregoing characteristics into consideration are well known to those of skill in the art and include (original residue: exemplary substitution): (Ala: Gly, Ser), (Arg: Lys), (Asn: Gin, His), (Asp: Glu, Cys, Ser), (Gln: Asn), (Glu: Asp), (Gly: Ala), (His: Asn, Gin), (Ile: Leu, Val), (Leu: Ile, Val), (Lys: Arg), (Met: Leu, Tyr), (Ser: Thr), (Thr: Ser), (Tip: Tyr), (Tyr: Trp, Phe), and (Val: Ile, Leu). Embodiments of this disclosure thus contemplate functional or biological equivalents of a peptide as set forth above. In particular, embodiments of the polypeptides can include variants having about 50%, 60%, 70%, 80%, 90%, and 95% sequence identity to the polypeptide of interest.

A peptide is obtained by any of various methods known in the art illustratively including isolation from a cell or organism, chemical synthesis, expression of a nucleic acid and partial hydrolysis of proteins. Chemical methods of peptide synthesis are known in the art and include solid phase peptide synthesis and solution phase peptide synthesis for instance. A peptide included in an inventive composition may be a naturally occurring or non-naturally occurring peptide. The term “naturally occurring” refers to a peptide endogenous to a cell, tissue or organism and includes allelic variations. A non-naturally occurring peptide is synthetic or produced apart from its naturally associated organism or modified and is not found in an unmodified cell, tissue or organism.

As used herein, the term “sample” is defined as a sample obtained from a biological organism, a tissue, cell, cell culture medium, or any medium suitable for mimicking biological conditions, or from the environment. Non-limiting examples include, saliva, gingival secretions, cerebrospinal fluid, gastrointestinal fluid, mucous, urogenital secretions, synovial fluid, cerebrospinal fluid, blood, serum, plasma, urine, cystic fluid, lymph fluid, ascites, pleural effusion, interstitial fluid, intracellular fluid, ocular fluids, seminal fluid, mammary secretions, vitreal fluid, nasal secretions, water, air, gas, powder, soil, biological waste, feces, cell culture media, cytoplasm, cell releasate, cell lysate, buffers, or any other fluid or solid media.

A peptide is illustratively recombinant. An inventive peptide may be co-expressed with associated tags, modifications, other proteins such as in a fusion peptide, or other modifications or combinations recognized in the art. Illustrative tags include 6×His, FLAG, biotin, ubiquitin, SUMO, or other tag known in the art. A tag is illustratively cleavable such as by linking to a peptide via an enzyme cleavage sequence that is cleavable by an enzyme known in the art illustratively including, but not limited to Factor Xa, thrombin, SUMOstar protein as obtainable from Lifesensors, Inc., Malvern, Pa., or trypsin. It is further appreciated that chemical cleavage is similarly operable with an appropriate cleavable linker.

Peptide expression is illustratively accomplished from transcription of nucleic acid sequence encoding a peptide and translation of RNA transcribed from the nucleic acid sequence. Peptide expression is optionally performed in a cell based system such as in E. coli, Hela cells, or Chinese hamster ovary cells. It is appreciated that cell-free expression systems are similarly operable.

It is recognized that numerous variants, including analogues or homologues, are within the scope of a peptide as defined herein according to some embodiments including amino acid substitutions, alterations, modifications, or other amino acid changes that increase, decrease, or do not alter the function of the ability of peptide to reduce or eliminate antibody induced enhancement of LF or LTx activity. It is appreciated that a variant includes one or more amino acid insertions, deletions, substitutions, or modifications.

Also provided are antibodies that enhance LT activity, LTx activity, or both in an assay system. An illustrative example of one such antibody is discussed in Boyer et al., Anal. Chem., 2007; 79 (22):8463-8470. A specific illustrative example of an activity enhancing antibody is AVR1674. Antibodies with the activity of AVR1674 are particularly preferred. One of ordinary skill in the art understands how to produce antibodies by standard techniques and screen the resulting monoclonal or polyclonal antibodies for their ability to interact with an epitope sequence. Such methods are illustratively taught by Monoclonal Antibodies: Methods and Protocols, Albitar, M, ed., Humana Press, 2010 (ISBN 1617376469); and Antibodies: A Laboratory Manual, Harlos, E, and Lane, D. eds., Cold Spring Harbor Laboratory Press, 1988 (ISBN-10: 0879693142).

Further aspects of the present disclosure concern the purification, otherwise termed isolation, and in particular embodiments, the substantial purification, of an encoded peptide, immunogen, or antibody. The term “purified” or “isolated” as used herein, is intended to refer to a composition, isolatable from other components, wherein the composition is purified to any degree relative to its naturally-obtainable state or state as expressed in a cell or synthetic system. A purified immunogen or peptide, therefore, also refers to a protein or peptide, free from the environment in which it may naturally occur.

Generally, “purified” or “isolated” will refer to a protein or peptide composition that has been subjected to fractionation to remove various other components, and which composition substantially retains its expressed biological activity. Where the term “substantially” purified is used, this designation will refer to a composition in which the protein or peptide forms the major component of the composition, such as constituting about 50% or more of the proteins in the composition.

Various methods for quantifying the degree of purification of the protein or peptide will be known to those of skill in the art in light of the present disclosure as based on knowledge in the art. These include, for example, determining the specific activity of an active fraction, or assessing the number of polypeptides within a fraction by SDS/PAGE analysis. A preferred method for assessing the purity of a fraction is to calculate the specific activity of the fraction, to compare it to the specific activity of the initial extract, and to thus calculate the degree of purity, herein assessed by a “-fold purification number”. The actual units used to represent the amount of activity will, of course, be dependent upon the particular assay technique chosen to follow the purification and whether or not the expressed protein or peptide exhibits a detectable activity.

Various techniques suitable for use in protein or peptide purification will be well known to those of skill in the art. These include, for example, precipitation with ammonium sulphate, polyethylene glycol, antibodies and the like or by heat denaturation, followed by centrifugation; chromatography steps such as ion exchange, gel filtration, reverse phase, hydroxylapatite and affinity chromatography; isoelectric focusing; gel electrophoresis; and combinations of such and other techniques. As is generally known in the art, it is believed that the order of conducting the various purification steps may be changed, or that certain steps may be omitted, and still result in a suitable method for the preparation of a substantially purified protein or peptide.

There is no general requirement that the protein or peptide always be provided in their most purified state. Indeed, it is contemplated that less substantially purified products will have utility in certain embodiments. Partial purification may be accomplished by using fewer purification steps in combination, or by utilizing different forms of the same general purification scheme. For example, it is appreciated that a cation-exchange column chromatography performed utilizing an HPLC apparatus will generally result in a greater-fold purification than the same technique utilizing a low pressure chromatography system. Methods exhibiting a lower degree of relative purification may have advantages in total recovery of protein product, or in maintaining the activity of an expressed protein.

It is known that the migration of a peptide can vary, sometimes significantly, with different conditions of SDS/PAGE (Capaldi et al., Biochem. Biophys. Res. Comm., 76:425, 1977). It will therefore be appreciated that under differing electrophoresis conditions, the apparent molecular weights of purified or partially purified expression products may vary.

Isolation of a peptide is included in some embodiments. Methods of peptide isolation illustratively include column chromatography, affinity chromatography, gel electrophoresis, filtration, or other methods known in the art. In some embodiments, peptide is expressed with a tag operable for affinity purification. A preferred tag is a 6×His tag. A 6×His tagged inventive protein is illustratively purified by Ni-NTA column chromatography or using an anti-6×His tag antibody fused to a solid support. (Geneway Biotech, San Diego, Calif.) Other tags and purification systems are similarly operable.

It is appreciated that a peptide is optionally not tagged. In such embodiments purification is optionally achieved by methods known in the art illustratively including ion-exchange chromatography, affinity chromatography, precipitation with salt such as ammonium sulfate, streptomycin sulfate, or protamine sulfate, reverse phase chromatography, size exclusion chromatography such as gel exclusion chromatography, HPLC, immobilized metal chelate chromatography, or other methods known in the art. One of skill in the art may select the most appropriate isolation and purification techniques without departing from the scope of this invention.

A peptide is illustratively recombinant. A peptide may be co-expressed with associated tags, modifications, other proteins such as in a fusion peptide, or other modifications or combinations recognized in the art. Illustrative tags include 6×His, FLAG, biotin, ubiquitin, SUMO, or other tag known in the art. A tag is illustratively cleavable such as by linking to PA immunogen or an associated protein via an enzyme cleavage sequence that is cleavable by an enzyme known in the art illustratively including Factor Xa, thrombin, SUMOstar protein as obtainable from Lifesensors, Inc., Malvern, Pa., or trypsin. It is further appreciated that chemical cleavage is similarly operable with an appropriate cleavable linker.

Protein expression is illustratively accomplished from transcription of PA immunogen nucleic acid sequence, translation of RNA transcribed from PA nucleic acid sequence, modifications thereof, or fragments thereof. Protein expression is optionally performed in a cell based system such as in E. coli, Hela cells, or Chinese hamster ovary cells. It is appreciated that cell-free expression systems are similarly operable.

A peptide is optionally chemically synthesized. Methods of chemical synthesis have produced proteins greater than 600 amino acids in length with or without the inclusion of modifications such as glycosylation and phosphorylation. Methods of chemical protein and peptide synthesis illustratively include solid phase protein chemical synthesis. Illustrative methods of chemical protein synthesis are reviewed by Miranda, L P, Peptide Science, 2000, 55:217-26 and Kochendoerfer G G, Curr Opin Drug Discov Devel. 2001; 4(2):205-14.

A peptide is optionally characterized by measurements including, without limitation, western blot, macromolecular mass determinations by biophysical determinations, SDS-PAGE/staining, HPLC and the like, antibody recognition assays, cell viability assays, apoptosis assays, assays for the activity of LF or LTx such as those described by Boyer et al., Anal. Chem., 2007; 79 (22):8463-8470, and assays to infer immune protection or immune pathology by adoptive transfer of cells, proteins or antibodies.

Also provided are isolated polynucleotides encoding a peptide that reduces or eliminates antibody enhancement of LF or LTx, or that encode an immunogen. These polynucleotides can be used to produce the peptides. It is appreciated that the degenerate nucleic acid code is well understood such that one of skill in the art fully and immediately understands a nucleic acid sequence that will produce a desired peptide sequence.

The term “nucleotide” is intended to mean a base-sugar-phosphate combination either natural or synthetic, linear, circular and sequential arrays of nucleotides and nucleosides, e.g. cDNA, genomic DNA, mRNA, and RNA, oligonucleotides, oligonucleosides, and derivatives thereof. Included in this definition are modified nucleotides which include additions to the sugar-phosphate groups as well as to the bases.

The term “nucleic acid” or “polynucleotide” refers to multiple nucleotides attached in the form of a single or double stranded molecule that can be natural, or derived synthetically, enzymatically, and by cloning methods. The term “oligonucleotide” refers to a polynucleotide of less than 200 nucleotides. The terms “nucleic acid” and “oligonucleotide” may be used interchangeably in this application.

A polynucleotide as used herein refers to single- or double-stranded molecules that may be DNA, including of the nucleotide bases A, T, C and G, or RNA, comprised of the bases A, U (substitutes for T), C, and G. The nucleic acid may represent a coding strand or its complement. Nucleic acids may be identical in sequence to the sequence naturally occurring or may include alternative codons that encode the same amino acid as that found in the naturally occurring sequence. Furthermore, nucleic acids may include codons that represent conservative substitutions of amino acids as are well known in the art.

The nucleic acid encoding the peptide of this invention can be part of a recombinant nucleic acid construct comprising any combination of restriction sites and/or functional elements as are well known in the art that facilitate molecular cloning and other recombinant DNA manipulations. Thus, the present invention further provides a recombinant nucleic acid construct comprising a nucleic acid encoding a peptide and/or polypeptide of this invention.

The present invention also provides a vector with a nucleic acid sequence encoding peptide sequence therein. Illustrative vectors include a plasmid, cosmid, cationic lipids, non-liposomal cationic vectors, cationic cyclodextrin, viruses with RNA or DNA genetic material, polyethylenimines, histidylated polylysine, or other vector system known in the art. A vector is optionally a plasmid. A suitable vector optionally possesses cell type specific expression or other regulatory sequences or sequences operable to stimulate or inhibit gene or protein expression. A vector illustratively contains a selection marker such as an antibiotic resistance gene.

An inventive nucleic acid sequence is provided. A nucleic acid sequence optionally encodes a peptide of SEQ ID NO: 1, SEQ ID NO: 2, SEQ ID NO: 3, other sequences herein, fragments thereof, or variants thereof such as immunogens and other peptides as described herein. The genetic code is a degenerate code whereby specific nucleic acid sequences encode for particular amino acids. As such it is well within the level of those of skill in the art to determine a nucleic acid sequence that will encode the inventive peptides.

The inventive nucleic acid sequence is optionally isolated from the cellular materials with which it is naturally associated. As used herein, the term “isolated nucleic acid” means a nucleic acid separated or substantially free from at least some of the other components of the naturally occurring organism, for example, the cell structural components commonly found associated with nucleic acids in a cellular environment and/or other nucleic acids. The isolation of nucleic acids is optionally accomplished by techniques such as cell lysis followed by phenol plus chloroform extraction, followed by ethanol precipitation of the nucleic acids. The nucleic acids of this invention can be isolated from cells according to methods well known in the art for isolating nucleic acids. Alternatively, the nucleic acids of the present invention can be synthesized according to standard protocols well described in the literature for synthesizing nucleic acids. Modifications to the nucleic acids of the invention are also contemplated, provided that the essential structure and function of the peptide or polypeptide encoded by the nucleic acid are maintained.

Numerous methods are known in the art for the synthesis and production of nucleic acid sequences illustratively including cloning and expression in cells such as E. coli, insect cells such as Sf9 cells, yeast, and mammalian cell types such as Hela cells, Chinese hamster ovary cells, or other cells systems known in the art as amendable to transfection and nucleic acid and/or protein expression. Methods of nucleic acid isolation are similarly recognized in the art. Illustratively, plasmid DNA amplified in E. coli is cleaved by suitable restriction enzymes such as NdeI and XhoI to linearize DNA. The DNA is subsequently isolated following gel electrophoresis using a S.N.A.P.™ UV-Free Gel Purification Kit (Invitrogen, Carlsbad, Calif.) as per the manufacturer's instructions.

Numerous agents are amenable to facilitate cell transfection illustratively including synthetic or natural transfection agents such as LIPOFECTIN, baculovirus, naked plasmid or other DNA, or other systems known in the art.

The nucleotide sequences of the invention may be isolated by conventional uses of polymerase chain reaction or cloning techniques such as those described in conventional texts. For example, the nucleic acid sequences of this invention may be prepared or isolated from DNA using DNA primers and probes and PCR techniques. Alternatively, the inventive nucleic acid sequence may be obtained from gene banks derived from Bacillus anthracis whole genomic DNA. These sequences, fragments thereof, modifications thereto and the full-length sequences may be constructed recombinantly using conventional genetic engineering or chemical synthesis techniques or PCR, and the like.

Also provided is a vector with a nucleic acid sequence encoding an immunogen or other peptide sequence therein. Illustrative vectors include a plasmid, cosmid, cationic lipids, non-liposomal cationic vectors, cationic cyclodextrin, viruses with RNA or DNA genetic material, polyethylenimines, histidylated polylysine, or other vector system known in the art. A vector is optionally a plasmid. A suitable vector optionally possesses cell type specific expression or other regulatory sequences or sequences operable to stimulate or inhibit gene or protein expression. A vector illustratively contains a selection marker such as an antibiotic resistance gene.

Also provided is a host cell transformed with an appropriate vector or with the inventive peptide sequence. Illustrative host cells include E. coli or Sf9 cells. Optionally, cell transfection is achieved by electroporation.

Recombinant or non-recombinant proteinase peptides or recombinant or non-recombinant proteinase inhibitor peptides or other non-peptide proteinase inhibitors can also be used in the present invention. Proteinase inhibitors are optionally modified to resist degradation, for example degradation by digestive enzymes and conditions. Techniques for the expression and purification of recombinant proteins are known in the art (see Sambrook Eds., Molecular Cloning: A Laboratory Manual 3′ ed. (Cold Spring Harbor, N. Y. 2001).

Some embodiments of the present invention are compositions containing nucleic acid sequences that can be expressed as encoded peptides. The engineering of DNA segment(s) for expression in a prokaryotic or eukaryotic system may be performed by techniques generally known to those of skill in recombinant expression. It is believed that virtually any expression system may be employed in the expression of the claimed nucleic acid and amino acid sequences.

Generally speaking, it may be more convenient to employ as the recombinant polynucleotide a cDNA version of the polynucleotide. It is believed that the use of a cDNA version will provide advantages in that the size of the gene will generally be much smaller and more readily employed to transfect the targeted cell than will a genomic gene, which will typically be up to an order of magnitude larger than the cDNA gene.

As used herein, the terms “engineered” and “recombinant” cells are synonymous with “host” cells and are intended to refer to a cell into which an exogenous DNA segment or gene, such as a cDNA or gene has been introduced. Therefore, engineered cells are distinguishable from naturally occurring cells which do not contain a recombinantly introduced exogenous DNA segment or gene. A host cell is optionally a naturally occurring cell that is transformed with an exogenous DNA segment or gene or a cell that is not modified. Engineered cells are cells having a gene or genes introduced through the hand of man. Recombinant cells include those having an introduced cDNA or genomic DNA, and also include genes positioned adjacent to a promoter not naturally associated with the particular introduced gene.

To express a recombinant encoded polypeptide in accordance with the present invention one would prepare an expression vector that comprises a polynucleotide under the control of one or more promoters. To bring a coding sequence “under the control of” a promoter, one positions the 5′ end of the translational initiation site of the reading frame generally between about 1 and 50 nucleotides “downstream” of (i.e., 3′ of) the chosen promoter.

The “upstream” promoter stimulates transcription of the inserted DNA and promotes expression of the encoded recombinant protein. This is the meaning of “recombinant expression” in the context used here.

Many standard techniques are available to construct expression vectors containing the appropriate nucleic acids and transcriptional/translational control sequences in order to achieve protein or peptide expression in a variety of host-expression systems. Cell types available for expression include, but are not limited to, bacteria, such as E. coli and B. subtilis transformed with recombinant phage DNA, plasmid DNA or cosmid DNA expression vectors.

Certain examples of prokaryotic hosts are E. coli strain RR1, E. coli LE392, E. coli B, E. coli. chi. 1776 (ATCC No. 31537) as well as E. coli W3110 (F-, lambda-, prototrophic, ATCC No. 273325); bacilli such as Bacillus subtilis; and other enterobacteriaceae such as Salmonella typhimurium, Serratia marcescens, and various Pseudomonas species.

In general, plasmid vectors containing replicon and control sequences that are derived from species compatible with the host cell are used in connection with these hosts. The vector ordinarily carries a replication site, as well as marking sequences that are capable of providing phenotypic selection in transformed cells. For example, E. coli is often transformed using pBR322, a plasmid derived from an E. coli species. Plasmid pBR322 contains genes for ampicillin and tetracycline resistance and thus provides easy means for identifying transformed cells. The pBR322 plasmid, or other microbial plasmid or phage must also contain, or be modified to contain, promoters that can be used by the microbial organism for expression of its own proteins.

In addition, phage vectors containing replicon and control sequences that are compatible with the host microorganism can be used as transforming vectors in connection with these hosts. For example, the phage lambda may be utilized in making a recombinant phage vector that can be used to transform host cells, such as E. coli LE392.

Further useful vectors include plN vectors and pGEX vectors, for use in generating glutathione S-transferase (GST) soluble fusion proteins for later purification and separation or cleavage. Other suitable fusion proteins are those with-galactosidase, ubiquitin, or the like.

Promoters that are most commonly used in recombinant DNA construction include the β-lactamase (penicillinase), lactose and tryptophan (trp) promoter systems. While these are the most commonly used, other microbial promoters have been discovered and utilized, and details concerning their nucleotide sequences have been published, enabling those of skill in the art to ligate them functionally with plasmid vectors.

For expression in Saccharomyces, the plasmid YRp7, for example, is commonly used. This plasmid contains the trp1 gene, which provides a selection marker for a mutant strain of yeast lacking the ability to grow in tryptophan, for example ATCC No. 44076 or PEP4-1. The presence of the trp1 lesion as a characteristic of the yeast host cell genome then provides an effective environment for detecting transformation by growth in the absence of tryptophan.

Suitable promoting sequences in yeast vectors include the promoters for 3-phosphoglycerate kinase or other glycolytic enzymes, such as enolase, glyceraldehyde-3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phosphoglucose isomerase, and glucokinase. In constructing suitable expression plasmids, the termination sequences associated with these genes are also ligated into the expression vector 3′ of the sequence desired to be expressed to provide polyadenylation of the mRNA and termination.

Other suitable promoters, which have the additional advantage of transcription controlled by growth conditions, include the promoter region for alcohol dehydrogenase 2, isocytochrome C, acid phosphatase, degradative enzymes associated with nitrogen metabolism, and glyceraldehyde-3-phosphate dehydrogenase, and enzymes responsible for maltose and galactose utilization.

In addition to microorganisms, cultures of cells derived from multicellular organisms may also be used as hosts. In principle, any such cell culture is operable, whether from vertebrate or invertebrate culture. In addition to mammalian cells, these include insect cell systems infected with recombinant virus expression vectors (e.g., baculovirus); and plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing one or more coding sequences.

In a useful insect system, Autographica californica nuclear polyhedrosis virus (AcNPV) is used as a vector to express foreign genes. The virus grows in Spodoptera fiugiperda cells. The isolated nucleic acid coding sequences are cloned into non-essential regions (for example the polyhedron gene) of the virus and placed under control of an AcNPV promoter (for example, the polyhedron promoter). Successful insertion of the coding sequences results in the inactivation of the polyhedron gene and production of non-occluded recombinant virus (i.e., virus lacking the proteinaceous coat coded for by the polyhedron gene). These recombinant viruses are then used to infect Spodoptera frugiperda cells in which the inserted gene is expressed (e.g., U.S. Pat. No. 4,215,051).

Examples of useful mammalian host cell lines are VERO and HeLa cells, Chinese hamster ovary (CHO) cell lines, W138, BHK, COS-7, 293, HepG2, NIH3T3, RIN and MDCK cell lines. In addition, a host cell may be chosen that modulates the expression of the inserted sequences, or modifies and processes the gene product in the specific fashion desired. Such modifications (e.g., glycosylation) and processing (e.g., cleavage) of protein products may be important for the function of the encoded protein.

Different host cells have characteristic and specific mechanisms for the post-translational processing and modification of proteins. Appropriate cell lines or host systems can be chosen to ensure the correct modification and processing of the foreign protein expressed. Expression vectors for use in mammalian cells ordinarily include an origin of replication (as necessary), a promoter located in front of the gene to be expressed, along with any necessary ribosome binding sites, RNA splice sites, polyadenylation site, and transcriptional terminator sequences. The origin of replication may be provided either by construction of the vector to include an exogenous origin, such as may be derived from SV40 or other viral (e.g., Polyoma, Adeno, VSV, BPV) source, or may be provided by the host cell chromosomal replication mechanism. If the vector is integrated into the host cell chromosome, the latter is often sufficient.

The promoters may be derived from the genome of mammalian cells (e.g., metallothionein promoter) or from mammalian viruses (e.g., the adenovirus late promoter, the vaccinia virus 7.5K promoter). Further, it is also possible, and may be desirable, to utilize promoter or control sequences normally associated with the desired gene sequence, provided such control sequences are compatible with the host cell systems.

A number of viral based expression systems may be utilized, for example, commonly used promoters are derived from polyoma, Adenovirus 2, cytomegalovirus and Simian Virus 40 (SV40). The early and late promoters of SV40 virus are useful because both are obtained easily from the virus as a fragment which also contains the SV40 viral origin of replication. Smaller or larger SV40 fragments may also be used, provided there is included the approximately 250 bp sequence extending from the HindIII site toward the BgI site located in the viral origin of replication.

In cases where an adenovirus is used as an expression vector, the coding sequences may be ligated to an adenovirus transcription/translation control complex, e.g., the late promoter and tripartite leader sequence. This chimeric gene may then be inserted in the adenovirus genome by in vino or in vivo recombination. Insertion in a non-essential region of the viral genome (e.g., region E1 or E3) will result in a recombinant virus that is viable and capable of expressing proteins in infected hosts.

Specific initiation signals may also be required for efficient translation of the claimed isolated nucleic acid coding sequences. These signals include the ATG initiation codon and adjacent sequences. Exogenous translational control signals, including the ATG initiation codon, may additionally need to be provided. One of ordinary skill in the art would readily be capable of determining this need and providing the necessary signals. It is well known that the initiation codon must be in-frame (or in-phase) with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements or transcription terminators.

In eukaryotic expression, one will also typically desire to incorporate into the transcriptional unit an appropriate polyadenylation site if one was not contained within the original cloned segment. Typically, the poly A addition site is placed about 30 to 2000 nucleotides “downstream” of the termination site of the protein at a position prior to transcription termination.

For long-term, high-yield production of recombinant proteins, stable expression is preferred. For example, cell lines that stably express constructs encoding proteins may be engineered. Rather than using expression vectors that contain viral origins of replication, host cells can be transformed with vectors controlled by appropriate expression control elements (e.g., promoter, enhancer, sequences, transcription terminators, polyadenylation sites, etc.), and a selectable marker. Following the introduction of foreign DNA, engineered cells may be allowed to grow for 1-2 days in an enriched medium, and then are switched to a selective medium. The selectable marker in the recombinant plasmid confers resistance to the selection and allows cells to stably integrate the plasmid into their chromosomes and grow to form foci, which in turn can be cloned and expanded into cell lines.

A number of selection systems may be used, including, but not limited, to the herpes simplex virus thymidine kinase, hypoxanthine-guanine phosphoribosyltransferase and adenine phosphoribosyltransferase genes, in tk-, hgprt- or aprt-cells, respectively. Also, antimetabolite resistance can be used as the basis of selection for dhfr, which confers resistance to methotrexate; gpt, which confers resistance to mycophenolic acid; neo, which confers resistance to the aminoglycoside G-418; and hygro, which confers resistance to hygromycin. It is appreciated that numerous other selection systems are known in the art that are similarly operable in the present invention.

It is contemplated that the isolated nucleic acids of the disclosure may be “overexpressed”, i.e., expressed in increased levels relative to its natural expression in cells of its indigenous organism, or even relative to the expression of other proteins in the recombinant host cell. Such overexpression may be assessed by a variety of methods, including radio-labeling and/or protein purification. However, simple and direct methods are preferred, for example, those involving SDS/PAGE and protein staining or immunoblotting, followed by quantitative analyses, such as densitometric scanning of the resultant gel or blot. A specific increase in the level of the recombinant protein or peptide in comparison to the level in natural human cells is indicative of overexpression, as is a relative abundance of the specific protein in relation to the other proteins produced by the host cell and, e.g., visible on a gel.

A nucleic acid of this invention can be in a cell, which can be a cell expressing the nucleic acid whereby a peptide and/or polypeptide of this invention is produced in the cell. In addition, the vector of this invention can be in a cell, which can be a cell expressing the nucleic acid of the vector whereby a peptide and/or polypeptide of this invention is produced in the cell. It is also contemplated that the nucleic acids and/or vectors of this invention can be present in a host animal (e.g., a transgenic animal) which expresses the nucleic acids of this invention and produces the peptides and/or polypeptides of this invention.

The nucleic acid encoding the peptides and polypeptides of this invention can be any nucleic acid that functionally encodes the peptides and polypeptides of this invention. To functionally encode the peptides and polypeptides (i.e., allow the nucleic acids to be expressed), the nucleic acid of this invention can include, for example, expression control sequences, such as an origin of replication, a promoter, an enhancer and necessary information processing sites, such as ribosome binding sites, RNA splice sites, polyadenylation sites and transcriptional terminator sequences.

Expression control sequences useful include promoters derived from metallothionine genes, actin genes, immunoglobulin genes, CMV, SV40, adenovirus, bovine papilloma virus, etc. A nucleic acid encoding a selected peptide or polypeptide can readily be determined based upon the genetic code for the amino acid sequence of the selected peptide or polypeptide and many nucleic acids will encode any selected peptide or polypeptide. Modifications in the nucleic acid sequence encoding the peptide or polypeptide are also contemplated. Modifications that can be useful are modifications to the sequences controlling expression of the peptide or polypeptide to make production of the peptide or polypeptide inducible or repressible as controlled by the appropriate inducer or repressor. Such methods are standard in the art. The nucleic acid of this invention can be generated by means standard in the art, such as by recombinant nucleic acid techniques and by synthetic nucleic acid synthesis or in vitro enzymatic synthesis.

Antibodies generated against or binding to a peptide of this invention are useful as research tools, clinical diagnostic assay components, or other uses. An antibody optionally is used as a screening agent for the presence or absence of LT or LTx in a sample. For example, an antibody enhances LT or LTx activity toward a particular substrate. This allows for detection of lower amounts of LT or LTx in a sample than would otherwise be achievable. The terms “antibody” and “antibodies” as used herein include monoclonal antibodies, polyclonal, chimeric, single chain, bispecific, simianized, and humanized antibodies, as well as Fab fragments, including the products of a Fab immunoglobulin expression library. The term “antigen” refers to the LT protein or any portion of the LT protein that includes residues 677-682 of immature LT protein. One particular example of an antibody suitable for use in processes for detecting LT or screening for LT activity is AVR1674 or AVR1675 (Boyer et al., Anal. Chem., 2007; 79 (22):8463-8470).

Antibodies as used herein can be polyclonal or monoclonal. An intact antibody, a fragment thereof (e.g., Fab or F(ab′)2), or an engineered variant thereof (e.g., sFv) can also be used. Such antibodies can be of any immunoglobulin class including IgG, IgM, IgE, IgA, IgD and any subclass thereof.

The antibodies provided herein can be monoclonal or polyclonal, optionally a F(ab)′² fragment lacking the Fc portion of the antibody. The antibodies can be prepared by generating B cell hybridomas, or by using laboratory animals such as mouse, humanized mouse, rat, rabbit or goat that are immunized with the peptides and/or polypeptides of this invention. The peptides and/or polypeptides optionally contain deletion, insertion and/or substitution mutations. Screening can then be carried out to identify antibodies that reduce LT activity such as the assays described in Boyer et al., Anal. Chem., 2007; 79 (22):8463-8470.

Monoclonal antibodies are generated by methods well known to those skilled in the art. An illustrative method is a modified version of the method of Kearney et al., J. Immunol. 123:1548-1558 (1979). Briefly, animals such as mice or rabbits are inoculated with the immunogen in adjuvant, and spleen cells are harvested and mixed with a myeloma cell line. The cells are induced to fuse by the addition of polyethylene glycol. Hybridomas are chemically selected by plating the cells in a selection medium containing hypoxanthine, aminopterin and thymidine (HAT). Hybridomas are subsequently screened for the ability to produce monoclonal antibodies that enhance LT or LTx activity. Hybridomas producing antibodies are cloned, expanded and stored frozen for future production.

Techniques for the production of single chain antibodies are known to those skilled in the art and described in U.S. Pat. No. 4,946,778 and can be used to produce single chain antibodies to the motifs described herein. Phage display technology may be used to select antibody genes having binding activities for the peptides of interest.

The antibodies are useful for enhancing LT or LTx activity. The antibody is optionally modified so that it is “humanized” by transplanting the complementarity determining regions of the hybridoma-derived antibody into a human monoclonal antibody as described by Jones et al., Nature, 32: 1522-525 (1986).

Polyclonal antibodies can be obtained by immunizing donors with vaccines or immunogens that induce antibodies that enhance the activities of LT or LTx, and/or have other important biologic functions, e.g., neutralizing antibodies. Serum from the selected donors is then pooled and made into immunoglobulin preparations.

An immunogen of the present invention may also optionally be modified to increase its immunogenicity. In a non-limiting example, the immunogen (antigen) may be coupled to chemical compounds or immunogenic carriers, provided that the coupling does not interfere with the desired biological activity of either the antigen or the carrier. For a review of some general considerations in coupling strategies, see Antibodies, A Laboratory Manual, Cold Spring Harbor Laboratory, ed. E. Harlow and D. Lane (1988). Useful immunogenic carriers known in the art, include, without limitation, keyhole limpet hemocyanin (KLH); bovine serum albumin (BSA), ovalbumin, PPD (purified protein derivative of tuberculin); red blood cells; tetanus toxoid; cholera toxoid; agarose beads; activated carbon; or bentonite. Useful chemical compounds for coupling include, without limitation, dinitrophenol groups and arsonilic acid.

The immunogen may also be modified by other techniques illustratively including denaturation with heat and/or SDS.

An immunogen may also be used in the form of a pharmaceutically acceptable salt. Suitable acids and bases which are capable of forming salts with the polypeptides of the present invention are well known to those of skill in the art, and include inorganic and organic acids and bases.

In another aspect, the invention provides a therapeutic composition and methods for treating humans and/or animals with anthrax infection. The therapeutic composition contains an immunogen or a peptide of the sequence (T/S)-X₁-(K/R)-(D/E)-X₂-X₃ or (TIS)-X₁-(K/R)-(D/E), nucleic acid sequence encoding a peptide of (T/S)-X₁-(K/R)-(D/E)-X₂-X₃ or (T/S)-X₁-(K/R)-(DE), or variant thereof as described herein and a suitable pharmaceutical carrier. Suitable pharmaceutically acceptable carriers facilitate administration of the peptide but are physiologically inert and/or nonharmful.

Carriers may be selected by one of skill in the art. Exemplary carriers include sterile water or saline, lactose, sucrose, calcium phosphate, gelatin, dextran, agar, pectin, peanut oil, olive oil, sesame oil, and water. Additionally, the carrier or diluent may include a time delay material, such as glycerol monostearate or glycerol distearate alone or with a wax. In addition, slow release polymer formulations can be used.

Optionally, the peptide may be combined with conventional pharmaceutical ingredients, such as preservatives, or chemical stabilizers. Suitable ingredients operable herein include, for example, casamino acids, sucrose, gelatin, phenol red, N-Z amine, monopotassium diphosphate, lactose, lactalbumin hydrolysate, and dried milk.

Alternatively, or in addition other agents useful in treating anthrax infection may be combined with a peptide, e.g., antibiotics or immunostimulatory agents and cytokine regulation elements, are expected to be useful in reducing or eliminating disease symptoms. Such agents may operate in concert with the therapeutic compositions of this invention. The development of therapeutic compositions containing these agents is within the skill of one in the art in view of the teachings of this invention.

A method of treating or preventing a Bacillus anthracis infection in a subject is provided including administering to the subject an effective amount of peptide, or peptide-encoding nucleic acid of this invention and optionally an anti-viral composition. The anti-viral compositions optionally include small drug-like molecule inhibitors of Bacillus anthracis replication and infection: nucleoside analogs such as ribavarin; EICAR; Pyrazogrin; 3-deazaguanine; GR92938X; and LY253963. These inhibitors are targeted to inhibit inosine monophosphates dehydrogenase (IMPDH). Inhibitors targeted to inhibit virus adsorption and entry are also useful. Prominent among this class are polyoxometalates and CL387626 (Wyeth-Ayerst, Pearl River, N.Y.). Other examples of polyoxometalates are T118, Trimeris' benzathrone, BABIM and RD30028.

The peptides are optionally incorporated into a pharmaceutical carrier such as saline, dextrose, water, glycerol, ethanol, other therapeutic compounds, or combinations thereof. The formulation is appropriate for the desired mode of administration and may include other immune modifiers such as heparin. The composition may also contain other additional biologically inert ingredients such as flavorants, fillers, etc.

Suitable methods of administration include, but are not limited to, intramuscular, intravenous, intranasal, mucosal, via aerosol delivery or by any route that will result in contact of a peptide with an antibody. Other non-limiting examples of such routes of administration include oral, parenteral and transdermal.

In addition, the peptides can be used to screen antisera from hyperimmune patients from whom antibodies having a very high affinity for the peptides can be derived.

Proteins, peptides or polypeptides of this invention contain the sequence of (T/S)-X₁-(K/R)-(D/E)-X₂-X₃ or (T/S)-X₁-(K/R)-(D/E), other sequences disclosed herein, variants thereof, or fragments thereof. It is appreciated that a peptide optionally includes additional amino acids N-terminal, C-terminal, or both to the sequence of (T/S)-X₁-(K/R)-(D/E)-X₂-X₃ or (T/S)-X₁-(K/R)-(D/E).

An effective amount of the compositions of this invention ranges from nanograms/kg to milligram/kg amounts for young children and adults. Based on this range, equivalent dosages for lighter or heavier body weights can be determined. The dose should be adjusted to suit the individual to whom the composition is administered and will vary with age, weight and metabolism of the individual. The exact amount of the composition required will vary from subject to subject, depending on the species, age, weight and general condition of the subject, the particular peptide or polypeptide used, its mode of administration and the like. An appropriate amount can be determined by one of ordinary skill in the art using only routine experimentation given the teachings herein. One skilled in the art will realize that dosages are best optimized by the practicing physician and methods for determining dose amounts and regimens and preparing dosage forms are described, for example, in Remington's Pharmaceutical Sciences, (Martin, E. W., ed., latest edition), Mack Publishing Co., Easton, Pa.

According to the method of the invention, a human or other animal may be treated for anthrax infection by administering an effective amount of a peptide. A more specific example of an “effective amount” is optionally between about 0.05 to about 100 μg/kg of a peptide with proper dosage easily selectable by one of skill in the art. A suitable dosage may be about 1.0 mL of such an effective amount. When given parenterally, peptide compositions are generally injected in doses ranging from 1 μg/kg to 100 mg/kg per day, optionally at doses ranging from 0.1 mg/kg to 20 mg/kg per day. The treatment may continue by continuous infusion or intermittent injection or infusion, at the same, reduced or increased dose per day for, e.g., 1 to 3 days, and additionally as determined by the treating physician.

Such a composition may be administered 1-3 times per day over a 1 day to 12 week period. However, suitable dosage adjustments may be made by the attending physician or veterinarian depending upon the age, sex, weight and general health of the subject. Such a composition is optionally administered parenterally, optionally intramuscularly or subcutaneously. However, it may also be formulated to be administered by any other suitable route, including orally or topically.

A peptide is optionally administered associated with a delivery molecule. An illustrative example of a delivery molecule is a cell penetrating peptide (CPP) such as Tat peptide or other peptide known in the art. Such CPPs are illustratively reviewed by Sebbage V, Bioscience Horizons (2009) 2 (1): 64-72. Optionally, a delivery molecule is an antibody that can be used to specifically target and promote cell penetration of a therapeutic peptide. Optionally, a delivery molecule is a nucleic acid, or other molecule known to improve cell permeability of a therapeutic peptide. The administration of a peptide is optionally accomplished as a pharmaceutical composition including a peptide and a pharmaceutically acceptable diluent, adjuvant, or carrier. The peptide may be administered without or in conjunction with known surfactants or other therapeutic agents. A stable pharmaceutical composition containing a peptide optionally includes the peptide at a suitable concentration in citrate buffered saline (5 or 20 mM citrate, 150 mM NaCl, pH 5.0) comprising 0.1% by weight of poloxamer 188 (Pluronic F-68, BASF Wyandotte, Parsippany, N.J.) and 0.002% by weight of polysorbate 80 (Tween 80, ICI Americas Inc., Wilmington, Del.). Another stable pharmaceutical composition containing a peptide includes the peptide at a desired concentration in 5 mM citrate, 150 mM NaCl, 0.2% poloxamer 188 and 0.002% polysorbate 80. Such combinations are described in U.S. Pat. Nos. 5,488,034 and 5,696,090 and corresponding International Publication No. WO 94/17819 (PCT/US94/01239). Peptide based constructs may be formulated like other known protein products or may be formulated in saline or a physiological buffer.

Various aspects of the present invention are illustrated by the following non-limiting examples. The examples are for illustrative purposes and are not a limitation on any practice of the present invention. It will be understood that variations and modifications can be made without departing from the spirit and scope of the invention. Reagents and materials illustrated herein are commonly cross reactive between mammalian species or alternative reagents with similar properties are commercially available, and a person of ordinary skill in the art readily understands where such reagents may be obtained.

### EXAMPLES

### Example 1

### Peptide Identification

Elucidation of the epitope for antibody AVR1674 and AVR1675 is performed by phage display analyses. The phage library includes 3×10⁹ different peptides and is constructed in M13mp19 bacteriophage vector. The variable insert is located four amino acid residues (via linker GGGS) from the amino terminus of protein III (pIII) and is encoded by ten NNK triplets, where N is any of the four natural DNA nucleotides (A, C, G or T) in equal mixtures and K is G or T in equal mixtures. Each NNK is thereby a mixture of 32 triplets that code for all 20 natural amino acids and one stop codon. The library (2.0×10¹¹ phage particles/ml) is selected using high binding 96-well microplate Cova-link, Nunc) coated overnight with target antibody AVR1674 (from hybridoma clone LFG2:4B10) in 100 mM bicarbonate buffer (pH 9.1). In the first round, 100 pmol target mAb is blocked with TBST/5% BSA for 1 hr/4° C. and washed 3-times with TBST. Target is panned against phage in 150 μl of TBST (50 mM Tris-HCl, 150 mM NaCl, 0.5% Tween 20 (v/v), pH 7.5) at room temperature on a plate shaker for 1 h. Each sample is washed 7-times with 150 μl of TBST to eliminate non-specifically bound phage clones. Bound phages are eluted with 90 μl of 0.1 M Glycine/HCl (pH 2.2)/5% BSA for 10 min at room temperature. Eluates are neutralized with 15 μl of 2 M Tris, pH 9.1. The eluates (90 μl) are amplified by infecting 20 mL of E. coli ER2738 cells in early logarithmic phase in Luria-Bertani (LB) medium for 4-5 hrs at 37° C. on a shaker in the presence of 20 μg/ml tetracycline. E. coli cells are cooled to 4° C. and centrifuged at 12,000×g for 15 min at 4° C. Supernatants are removed and phage enriched twice by precipitation with 0.25 vols of PEG/NaCl (16.7% (w/v) polyethylene glycol 8000, 2.5 M NaCl) and alternating TBS solubilization and high-speed centrifugation to remove bacterial debris. This selection procedure is repeated two more times with amplified phage input consistently at 2.0×10¹¹ phage particles/ml from the preceding selection. Target mAb is reduced to 40 pmol in the second panning and 10 pmol in the third panning to increase stringency for binding. Phage is titered by standard titration and spectroscopic methods (Smith G. P. and Scott, J. K., 1993. Libraries of peptides and proteins displayed on filamentous phage. Methods Enzymol. 217, p. 228-257). For individual clone selection, E. coli cells are infected with phage from the third eluate at dilution between 1×10⁸⁻¹⁰ and 10 single phage colonies (blue screen) are randomly selected. The selected clones are amplified in 20 mL of LB containing 20 μg/ml tetracycline in sterile flasks. Each clone is characterized by ELISA and ssDNA extraction.

The ability of each clone to interact with AVR1674 and 1675 is studied by ELISA. AVR1674 mAb is dissolved in carbonate buffer (150 μl/well) to the wells of a 96-well microtiter plate (Cova-link, Nunc) overnight at 4° C. Corresponding molar amounts of other antibodies are coated under the same conditions described above. The wells are washed four times with wash buffer PBST (50 mM sodium phosphate, 150 mM NaCl, 0.5% Tween 20 (v/v), pH 7.5) and incubated in 150 μl of blocking buffer (PBST/5% goat serum) for 1 hr at 4° C. Amplified phage particles are diluted in 150 μl/well of PBST, in triplicate, and incubated for 1 h at room temperature on a shaker. The wells are washed five times with PBST. HRP-labeled sheep anti-M13 IgG in PBST (1:5000), 150 μl per well, is added and incubated for lhr at room temperature. Finally, the microtiter plates are washed six times with PBST (150 μl per well), followed by the addition of 100 μl substrate. Once color development is satisfactory, 100 μl of stop solution is added and mixed. Absorbance is monitored on a Spectramax 380 (Molecular Devices, Ca) plate reader at 450 nm.

Given the epitope similarity between both AVR clones, clone AVR1675/LFG2:3D10 is selected to pursue mapping. A heptapeptide library is subjected to three panning selections against pure IgG by solid-phase binding. In all selections, high affinity phage peptides are eluted by low pH briefly to improve viable phage recovery. Stringency is introduced into successive selection steps by lowering the target mAb concentration used in panning from 100 pmol to 10 pmol in the final panning selection. The input phage concentration remaines constant at 2×10¹¹ pfu/ml each round using prior eluent. Viable phage is calculated by titrating amplified phage into competent E. coli ER2258 and counting X-gal-positive blue plaques in an agar overlay assay (Barbas, C. F. 2001. Phage display: laboratory manual. Cold Spring Laboratory Press, Cold Spring Harbor, N.Y.) To generate a consensus sequence of high confidence, Pan 3 phage clones are isolated in greater number and ELISA-positive clones were sequenced.

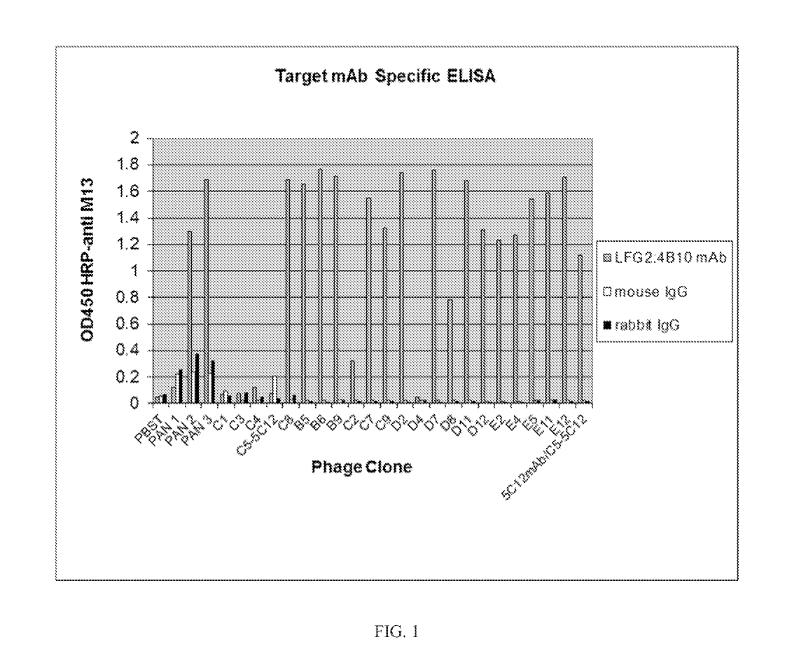

FIG. 1 summarizes the results of the target mAb ELISA. Typical enrichment between panning steps is observed. Clones from the third panning are isolated and tested for relative affinities to the target mAb. Sixteen clones achieve an OD≧0.5 (an arbitrary value defining high-affinity interaction) and show a 12-16-fold increase in signal from the un-enriched Pan 1. None of the clones react with mouse IgG or rabbit IgG above background eliminating non-specific protein interaction.

Successful clones are subjected to DNA sequencing analyses. Single-stranded DNA from individual phage clones is purified by NaI and ethanol precipitation. The DNA from the selected clones is amplified using the polymerase chain reaction (PCR), with the PCR protocol and necessary reagents provided with the Dye Terminator Cycle Sequencing Core Kit (PE Applied Biosystems, Foster City, Calif., USA). The primers used for the sequencing had the following nucleotide sequences: 5′-HO-GTA TGG GAT TTT GCT AAA CAA C-3′ (−28 gIII primer) (SEQ ID NO: 5) and 5′-HO-CCC TCA TAG TTA GCG TAA CG-3′ (−96 gIII primer) (SEQ ID NO: 6). The sequences are analyzed on an ABI Prism 377 DNA sequencer (Perkin Elmer, Foster City, Calif., USA).

DNA sequencing of all randomly selected individual clones from Pan 3 yields unambiguous sequences that permit read-through and translation of residues in the peptide insert and into pIII-fusion protein of M13 phage. The amino acid sequences are aligned by CLUSTAL-X in and are illustrated in Table 1.

TABLE 1

| Peptide | Sequence | SEQ ID NO: |
|---|---|---|
| 5c12-c5 | YAILEDH | 7 |
| AVR1674-C1 | NHHYSHL | 8 |
| AVR1674-C3 | LPLTPLP | 9 |
| AVR1674-C4 | SPEARHP | 10 |
| LF | THQDEIV | 11 |
| AVR1674-C8 | TFKDEIV | 12 |
| AVR1674-D12 | TFKDEIV | 12 |
| AVR1674-E4 | TFKDEIV | 12 |
| AVR1674-E11 | TFKDEIV | 12 |
| AVR1674-C9 | TFKDDIH | 13 |
| AVR1674-85 | TYKDDIR | 14 |
| AVR1674-D2 | TYKDDIR | 14 |
| AVR1674-C7 | TFKDDLF | 15 |
| AVR1674-D4 | TFKDDGY | 16 |
| AVR1674-B6 | TYLDDLY | 17 |
| AVR1674-D11 | TYLDDLY | 17 |
| AVR1674-E2 | TFLDDAP | 18 |
| AVR1674-D8 | TWRDDIP | 19 |
| AVR1674-E5 | TYRDDPP | 20 |
| AVR1674-C2 | TVLDDVA | 21 |
| AVR1674-D7 | TVRDDQI | 22 |
| AVR1674-B9 | TFRDEPM | 23 |
| AVR1674-E12 | TVRDEPL | 24 |

Four high affinity peptides (AVR1674-C8, -D12, -E4 and E11) share the identical sequence TFKDEIV. This includes clone (C8). Two additional phage clones with high affinities, clones AVR1674-B5 and -D2, share sequence TYKDDIR. The remaining clones with high affinities have identical or similar amino acids at conserved positions. In general, peptides are enriched with charged and polar amino acids (Thr, Lys, Arg, Asp, Glu) and aromatic/hydrophobic residues in the second and seventh positions. Two clones with significant reduction in binding to target mAb (AVR-C2 and -D4) have unique amino acid changes in critical positions. The substitution of Leu for a basic charge in p3 reduces ELISA signal by 80% while substituting a Gly for larger hydrophobic residues eliminates binding. Control (5C12-C5) and non-binding clones have poor alignment with 16 high affinity clones. The alignment generates a 6-residue consensus sequence identified as T×K/R D D/E y x/y (x=aromatic, y=non-polar, n=no preference). The LF primary sequence is aligned against this peptide and the sequence THQDEIY (SEQ ID NO: 11) (aa 677-682 of immature LF) is identified as best match. This sequence resides in a large ordered loop (L2) found in Domain IV of LF which is inserted between two β-sheet strands, 4β2 and 4β3, where it partly obscures the active site (Pannifer A D, Wong T Y, Schwarzenbacher R, Renatus M, Petosa C, Bienkowska J, Lacy D B, Collier R J, Park S, Leppla S H, Hanna P, Liddington R C. 2001. Crystal structure of the anthrax lethal factor. Nature. 2001 Nov. 8; 414(6860):229-33.). Upon closer examination of the LF solvent-exposed L2 (1J7N; RasMol) the most conserved residues of the putative epitope (Thr677, Asp680, Glu681) extends outward from the strands while the surrounding residues are oriented toward the interior of the molecule. These residues are thus coordinated in an exposed plane amenable to contact with CDR residues of the target antibody.

### Example 2

### Peptide Characterization

To further elucidate the core required peptide sequences necessary for binding AVR1674 and preventing its interaction with LF, synthetic peptides are designed against the predicted epitope in LF and reference phage clone AVR1674-C8. Testing the isolated peptide removes possibility of mAb binding via non-specific phage proteins. Additionally both phage and LF sequences can be measured accurately for relative and kinetic binding to target mAb. Peptide sequences are modifications of AVR1674-C8 and are listed in Table 2:

TABLE 2

| Peptide | SEQ ID NO: |
|---|---|
| TFKDEIGGGSK-biotin | 25 |
| AFKDEIGGESK-biotin | 26 |
| TAKDEIGGGSK-biotin | 27 |
| TFADEIGGGSK-biotin | 28 |
| TFKAEIGGGSK-biotin | 29 |
| TFKDAIGGGSK-biotin | 30 |
| TFKDEAGGGSK-biotin | 31 |
| AFKDEIGGGSK-biotin | 32 |
| TAKDEIGGGSK-biotin | 33 |
| TFADEIGGGSK-biotin | 34 |
| TFKAEIGGGSK-biotin | 35 |
| THKDEIGGGSK-biotin | 36 |
| TFQDEIGGGSK-biotin | 37 |
| THQDEIYEQK-biotin | 38 |

In addition to phage peptide (AVR1674-C8 TFKDEIGGGSK-bio) (SEQ ID NO: 25) two peptides are designed to provide a putative epitope of LF (THQDEIYEQK-bio) (SEQ ID NO: 38) and an extended LF peptide (mature LF₆₃₇₋₆₆₄ bio-PNIAEQYTHQDEIYEQVHSKGLYVPESR) (SEQ ID NO: 39), which has the entire L2 and 4β3 sequences to introduce possible structural elements. The ability of the peptides to bind to mAb AVR1674 is studied by competitive ELISA or biolayer interferometry.

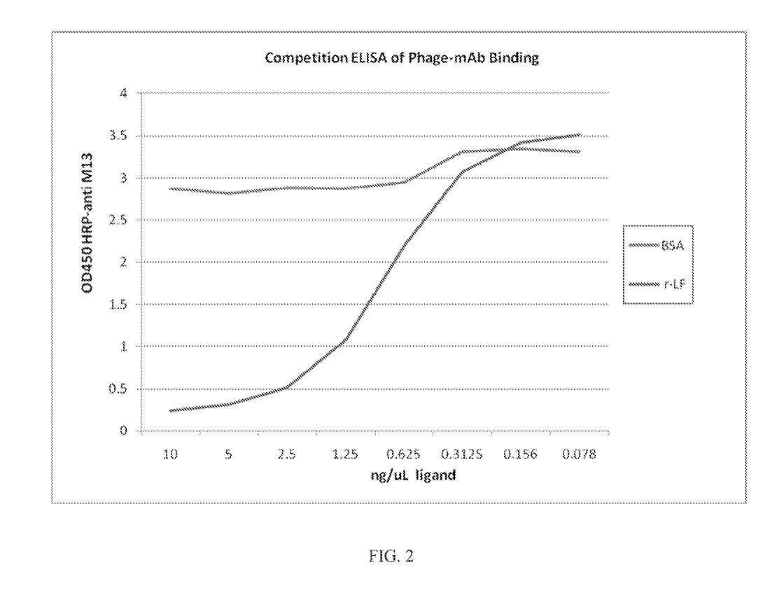

Each of the peptides of Table 2 is subjected to a competition assay using recombinant LF as the competing ligand. Recombinant LF at 0-10 μg/ml is pre-incubated with phage clones or isolated peptides (with or without biotinylation) for 10 min at room temperature. Phage and r-LF are diluted in 150 μl/well of PBST, in triplicate, and incubated for 1 h at room temperature on 96-well plate coated with 15 ug/ml mAb. The wells are washed five times with PBST. HRP-labeled sheep anti-M13 IgG in PBST (1:5000), 150 μl per well, is added and incubated for 1 hr at room temperature. ELISA is developed as discussed in Example 1. The inhibition of binding by the phage displaying peptide of AVR-1674 C8 is illustrated in FIG. 2. These results demonstrate that phage binds competitively with r-LF for the antibody binding domain. BSA has no effect in phage-mAb interaction.

For biolayer interferometry, isolated biotinylated peptides of Table 2 are synthesized. Octet Qke analysis is performed at temperature control at 30° C. in PBS buffer. Streptavidin (SA) sensors are pre-wet for 10 min in buffer prior to use and microplates used in the Octet are filled with 200 μl of sample or buffer and agitated at 700 rpm. SA-coated tips are saturated with 25 μg/ml biotinylated synthetic peptides. Typical capture levels are 0.70±0.15 nm within a row of eight tips with the standard deviation within the instrument noise. A nM titration of LFG2:4B10 mAb is bound for 500 s and allowed to dissociate for 500 s in PBS buffer. Dissociation buffer was used only once to prevent non-specific binding. Blank binding cycles containing only peptide are used to correct for baseline drift. Peptides are compared in the same experiment by coupling each onto its own tips in triplicate. Shift data from the Octet are exported for processing and analysis in Data Analysis 6.4. To deduce a direct binding affinity via the kinetic rate constants (KD=koff/kon, where KD=equilibrium dissociation rate constant, kon=association rate constant, and koff=dissociation rate constant) the buffer subtracted Octet data are fit globally to a simple 1:1 Langmuir model.

A significant improvement in binding response by nearly 7-fold (4.83 nm vs. 0.7 nm) and dissociation (13.9 nM vs. 53 nM) is exhibited using the phage peptide AVR1674-C8 (TFKDEIGGGSK-biotin: SEQ ID NO: 25) rather than native epitope (THQDETYEQK-biotin; SEQ ID NO: 38) (Table 3). The putative LF sequence is sufficient for binding the target mAb with no effect of sequence outside the THQDEIYEQ (SEQ ID NO: 40) epitope containing sequence in the L2. Furthermore, the discovered sequence (TFKDEI; SEQ ID NO: 4) in phage enhances binding through molecular interactions not present in the synthetic peptide structure and/or primary sequence. To obtain a more accurate kinetic determination of the phage peptide below saturation conditions, a titration of mAb LFG2 is tested against peptide in a follow-up experiment. A higher affinity (K_{D}=9.97 nM) is calculated with a 4-point titration curve. This approximates dissociation constants (K_{D}=≦10 nM) for anti-LF mAbs previously studied (data not shown). Similar studies are performed on all peptides of Table 2. The results are illustrated in Table 3.

TABLE 3

| | | Assoc. | | | |
|---|---|---|---|---|---|
| | | (Sample) | Conc. | Response | |
| Peptide | SEQ ID NO: | Loc. | (nM) | R | K_{D} (M) |
| TFKDEIGGGSK-biotin | 25 | A4 | 500 | 4.8387 | 1.39E-08 |
| AFKDEIGGGSK-biotin | 26 | B4 | 500 | 0.5352 | 3.51E-08 |
| TAKDEIGGGSK-biotin | 27 | C4 | 500 | 3.7854 | 3.15E-08 |
| TFADEIGGGSK-biotin | 28 | D4 | 500 | 0.4475 | 2.52E-08 |
| TFKAEIGGESK-biotin | 29 | E4 | 500 | 0.2612 | na |
| TFKDAIGGGSK-biotin | 30 | F4 | 500 | 1.0132 | 2.45E-08 |
| TFKDEAGGGSK-biotin | 31 | G4 | 500 | 5.2909 | 1.64E-08 |
| AFKDEIGGGSK-biotin | 32 | B4 | 500 | 0.556 | 2.87E-08 |
| TAKDEIGGGSK-biotin | 33 | C4 | 500 | 3.7743 | 2.09E-08 |
| TFADEIGGGSK-biotin | 34 | D4 | 500 | 0.4596 | 2.39E-08 |
| TFKAEIGGGSK-biotin | 35 | E4 | 500 | 0.2492 | n/a |
| THKDEIGGESK-biotin | 36 | F4 | 500 | 2.3704 | 0.00000006 |
| TFQDEIGGGSK-biotin | 37 | G4 | 500 | 1.3414 | 3.44E-08 |
| THQDEIYEQK-biotin | 38 | H4 | 500 | 0.7024 | 5.31E-08 |

### Example 3

### Inhibition of the Stimulation of LT Activity

The activity of LF is assayed in 40 μL total volume of reaction buffer (RB) containing 20 mM HEPES buffer pH 7.3, 1 mM DTT, 20 μM CaC12, 10 mM MgCl₂, 20 μM ZnCl2, protease inhibitor mix, and 2 nmol of substrate peptide LF-4 described in Boyer et al., Anal. Chem., 2007; 79 (22):8463-8470.

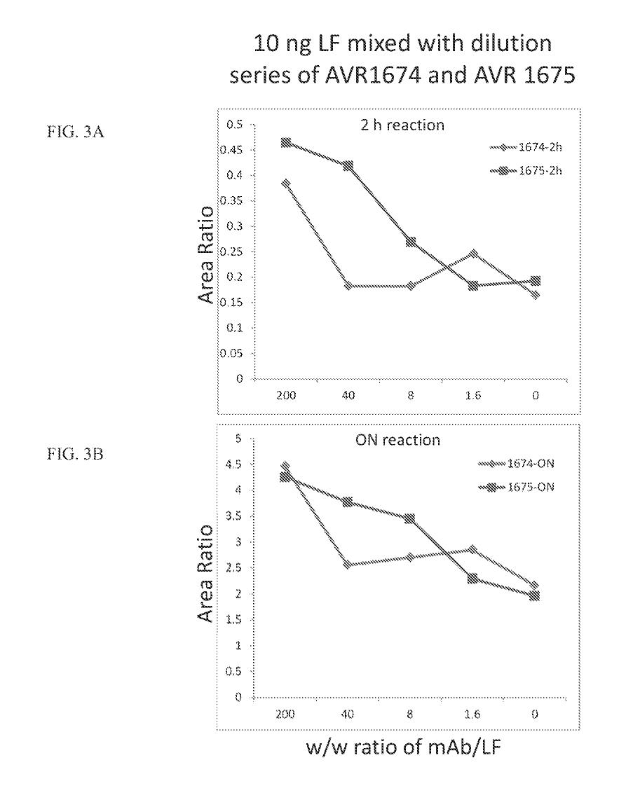

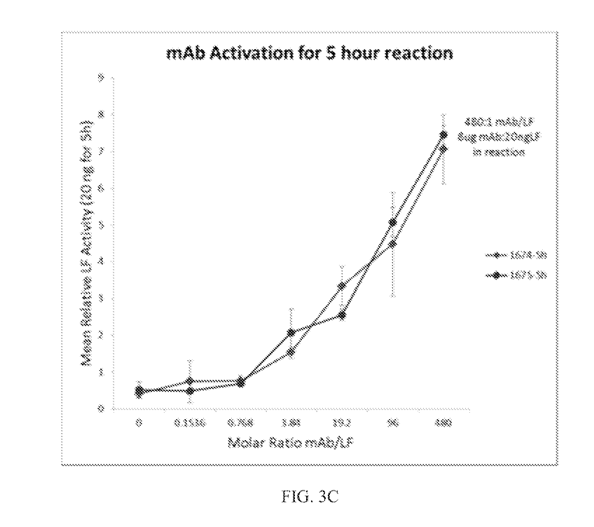

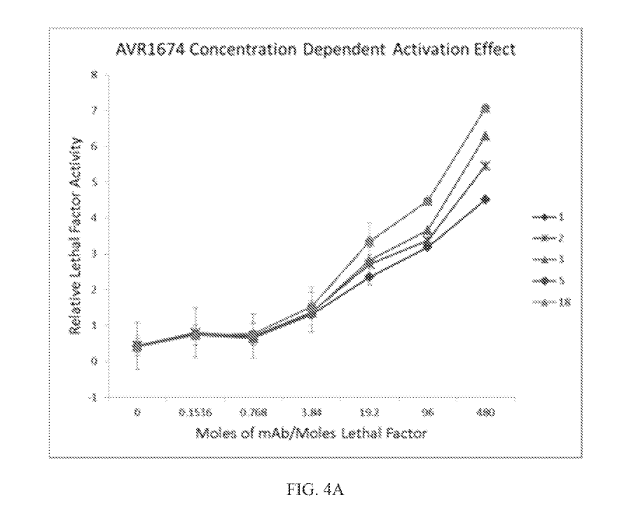

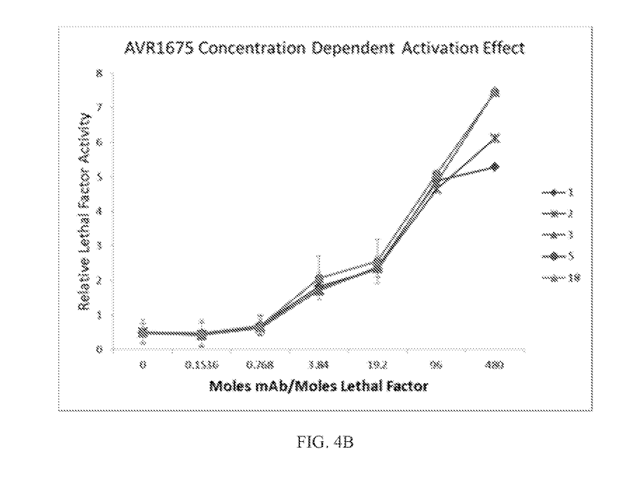

When assayed alone, both antibody AVR1674 and AVR1675 enhance the activity of LF. (FIG. 3) FIG. 3A illustrates a 2 hour incubation of antibody with LF prior to substrate cleavage. FIG. 3B illustrates a 16 hour incubation. FIG. 3C illustrates onset time of enhancement of LF activity with AVR1674 or AVR1675 following a 5 hour reaction demonstrating that the sequence of (T/S)-X₁-(K/R)-(D/E)-X₂-X₃ or (T/S)-X₁-(K/R)-(D/E) is essential for enhancing antibody induced activity. With increasing antibody to LF ratio, greater activity is observed. FIG. 4A illustrates a concentration dependent activation of LF by antibody AVR1674. FIG. 4B illustrates a concentration dependent activation of LF by antibody AVR1675.

For MALDI-TOF MS detection, a 1- or 2-μL aliquot of the LF reaction mixture is removed and added respectively to 9 or 18 μL of α-cyano-4-hydroxycinnamic acid (CHCA) at 5 mg/mL in 50% acetonitrile, 0.1% trifluoroacetic acid, and 1 mM ammonium phosphate (CHCA matrix), with or without 1 pmol of NT- and optionally CT-ISTD, mixed, and 0.5 μL is spotted in triplicate onto a 192-spot stainless steel MALDI plate (Applied Biosystems, Framingham, Mass.), and then mass spectra are collected from 750 to 3200 mass/charge (m/z) or as described, in MS positive ion reflectron mode on the Applied Biosystems 4700 Proteomics Analyzers (Framingham, Mass.). This instrument uses a nitrogen laser at 337 nm, and each final mass spectrum is an average of spectra obtained from 2400 laser shots.

LF in a sample is incubated with RB, antibody AVR1674, AVR1675, or both, and substrate in the presence or absence of each of the peptides of Table 2 at 37° C. for 2 h, and a 1-μL aliquot of the reaction is used for MALDI-TOF MS analysis. Each of the peptides of Table 2 reduces the activity of LF enhanced by antibody AVR1674.

Various modifications of the present invention, in addition to those shown and described herein, will be apparent to those skilled in the art of the above description. Such modifications are also intended to fall within the scope of the appended claims.

It is appreciated that all reagents are obtainable by sources known in the art unless otherwise specified. Methods of nucleotide amplification, cell transfection, and protein expression and purification are similarly within the level of skill in the art.

Patents and publications mentioned in the specification are indicative of the levels of those skilled in the art to which the invention pertains. These patents and publications are incorporated herein by reference to the same extent as if each individual application or publication was specifically and individually stated herein as incorporated herein by reference for the entirety of their teaching.

The foregoing description is illustrative of particular embodiments of the invention, but is not meant to be a limitation upon the practice thereof. The following claims, including all equivalents thereof, are intended to define the scope of the invention.

We claim:

1. A tagged peptide comprising from 4 to 100 amino acids, said peptide comprising the sequence (T/S)-X₁-(K/R)-(D/E)-X₂-X₃ wherein X₁ is phenylalanine, alanine, or histidine.

2-3. (canceled)

4. The peptide of claim 1 wherein the amino acid at position 4 is glutamate.

5. The peptide of claim 1 wherein X₃ is isoleucine or alanine.

6. The peptide of claim 1 wherein said peptide comprises the sequence of TFKDEI (SEQ ID NO: 4).

7. The peptide of claim 1, wherein said peptide consists of 6-15 amino acids.

8. The peptide of claim 1, wherein said peptide consists of 11 amino acids.

9. (canceled)

10. A process of reducing enhancement of B. anthracis lethal factor activity comprising:
combining lethal factor with a peptide, said peptide comprising the sequence (T/S)-X₁-(K/R)-(D/E)-X₂-X₃, wherein X₁ is phenylalanine, alanine, or histidine, said combining in an aqueous medium.

11. The process of claim 10 wherein said medium further comprises an antibody that recognizes an epitope comprising the sequence of THQDEIFEQK (SEQ ID NO: 41), or portion thereof.

12. The process of claim 11, wherein said antibody is AVR1674.

13. (canceled)

14. The process of claim 10 wherein the amino acid at position 4 is glutamate.

15. The process of claim 10 wherein the X₃ is isoleucine or alanine.

16. The process of claim 10 wherein said peptide comprises the sequence of TFKDEI (SEO ID NO: 4).

17. The process of claim 10 wherein said peptide consists of 6-15 amino acids.

18-20. (canceled)

21. An immunogen comprising:
a portion of B. anthracis lethal factor peptide, wherein said peptide comprises amino acid sequences from the two regions that flank residues 677-680, 677-686, or any portion or the whole of residues 667-688 representing the L2 region, said peptide excluding the sequence of residues 677-680 of SEQ ID NO: 2 or (T/S)-X₁-(K/R)-(D/E).

22. The immunogen of claim 21 wherein said immunogen comprises a portion of the lethal factor domain IV, said domain comprising a deletion relative to SEQ ID NO: 2, wherein at least one amino acid of residues 677-680 of SEQ ID NO: 2, optionally, the amino acid at P1, P3, P4, or combinations thereof is deleted.

23. The immunogen of claim 21 wherein said immunogen does not include a full length LF region.

24. The immunogen of claim 21 wherein said immunogen does not include any portion of the L2 region.

25. The immunogen of claim 21 wherein said immunogen comprises a portion of the lethal factor domain IV, said domain comprising an amino acid substitution of one or more of residues 677-680 of SEQ ID NO: 2 wherein P1 is neither T nor S, P3 is neither K nor R, P4 is neither D nor E, or combinations thereof.

26. The immunogen of claim 21 wherein said immunogen has a substitution of the entire L2 region of LF with a peptide of 2 or more amino acids, optionally said amino acids are alanine or glycine.

27. The immunogen of claim 21 wherein said immunogen does not perturb the structure of the regions flanking the L2 region of lethal factor.


SEQUENCE LISTING (41 sequences)

SEQ ID NO: 1 - protein, 10 aa, Artificial Sequence
  Synthetic peptide
THQDEIYEQV

SEQ ID NO: 2 - protein, 833 aa, Artificial Sequence
  Wild-type immature LF sequence
WILDTYPEIMMATRELFSEQENCEMNIKKEFIKVISMSCLVTAITLSGPVFIPLVQGAGGHGDVGMHVKEKEKNKDENKRKDEERNKTQEEHLKEIMKHIVKIEVKGEEAVKKEAAEKLLEKVPSDVLEMYKAIGGKIYIVDGDITKHISLEALSEDKKKIKDIYGKDALLHEHYVYAKEGYEPVLVIQSSEDYVENTEKALNVYYEIGKILSRDILSKINQPYQKFLDVLNTIKNASDSDGQDLLFTNQLKEHPTDFSVEFLEQNSNEVQEVFAKAFAYYIEPQHRDVLQLYAPEAFNYMDKFNEQEINLSLEELKDQRMLARYEKWEKIKQHYQHWSDSLSEEGRGLLKKLQIPIEPKKDDIIHSLSQEEKELLKRIQIDSSDFLSTEEKEFLKKLQIDIRDSLSEEEKELLNRIQVDSSNPLSEKEKEFLKKLKLDIQPYDINQRLQDTGGLIDSPSINLDVRKQYKRDIQNIDALLHQSIGSTLYNKIYLYENMNINNLTATLGADLVDSTDNTKINRGIFNEFKKNFKYSISSNYMIVDINERPALDNERLKWRIQLSPDTRAGYLENGKLILQRNIGLEIKDVQIIKQSEKEYIRIDAKVVPKSKIDTKIQEAQLNINQEWNKALGLPKYTKLITFNVHNRYASNIVESAYLILNEWKNNIQSDLIKKVTNYLVDGNGRFVFTDITLPNIAEQYTHQDEIYEQVHSKGLYVPESRSILLHGPSKGVELRNDSEGFIHEFGHAVDDYAGYLLDKNQSDLVTNSKKFIDIFKEEGSNLTSYGRTNEAEFFAEAFRLMHSTDHAERLKVQKNAPKTFQFINDQIKFIINS

SEQ ID NO: 3 - protein, 5 aa, Artificial Sequence
  Synthetic peptide
GGGSK

SEQ ID NO: 4 - protein, 6 aa, Artificial Sequence
  Synthetic peptide
TFKDEI

SEQ ID NO: 5 - DNA, 22 bp, Bacillus anthracis
gtatgggattttgctaaacaac

SEQ ID NO: 6 - DNA, 20 bp, Bacillus anthracis
ccctcatagttagcgtaacg

SEQ ID NO: 7 - protein, 7 aa, Artificial Sequence
  Synthetic peptide
YAILEDH

SEQ ID NO: 8 - protein, 7 aa, Artificial Sequence
  Synthetic peptide
NHHYSHL

SEQ ID NO: 9 - protein, 7 aa, Artificial Sequence
  Synthetic peptide
LPLTPLP

SEQ ID NO: 10 - protein, 7 aa, Artificial Sequence
  Synthetic peptide
SPEARHP

SEQ ID NO: 11 - protein, 7 aa, Artificial Sequence
  Synthetic peptide
THQDEIY

SEQ ID NO: 12 - protein, 7 aa, Artificial Sequence
  Synthetic peptide
TFKDEIV

SEQ ID NO: 13 - protein, 7 aa, Artificial Sequence
  Synthetic peptide
TFKDDIH

SEQ ID NO: 14 - protein, 7 aa, Artificial Sequence
  Synthetic peptide
TYKDDIR

SEQ ID NO: 15 - protein, 7 aa, Artificial Sequence
  Synthetic peptide
TFKDDLF

SEQ ID NO: 16 - protein, 7 aa, Artificial Sequence
  Synthetic peptide
TFKDDGY

SEQ ID NO: 17 - protein, 7 aa, Artificial Sequence
  Synthetic peptide
TYLDDLY

SEQ ID NO: 18 - protein, 7 aa, Artificial Sequence
  Synthetic peptide
TFLDDAP

SEQ ID NO: 19 - protein, 7 aa, Artificial Sequence
  Synthetic peptide
TWRDDIP

SEQ ID NO: 20 - protein, 7 aa, Artificial Sequence
  Synthetic peptide
TYRDDPP

SEQ ID NO: 21 - protein, 7 aa, Artificial Sequence
  Synthetic peptide
TVLDDVA

SEQ ID NO: 22 - protein, 7 aa, Artificial Sequence
  Synthetic peptide
TVRDDQI

SEQ ID NO: 23 - protein, 7 aa, Artificial Sequence
  Synthetic peptide
TFRDEPM

SEQ ID NO: 24 - protein, 7 aa, Artificial Sequence
  Synthetic peptide
TVRDEPL

SEQ ID NO: 25 - protein, 11 aa, Artificial Sequence
  Synthetic peptide
TFKDEIGGGSK

SEQ ID NO: 26 - protein, 11 aa, Artificial Sequence
  Synthetic peptide
AFKDEIGGGSK

SEQ ID NO: 27 - protein, 11 aa, Artificial Sequence
  Synthetic peptide
TAKDEIGGGSK

SEQ ID NO: 28 - protein, 11 aa, Artificial Sequence
  Synthetic peptide
TFADEIGGGSK

SEQ ID NO: 29 - protein, 11 aa, Artificial Sequence
  Synthetic peptide
TFKAEIGGGSK

SEQ ID NO: 30 - protein, 11 aa, Artificial Sequence
  Synthetic peptide
TFKDAIGGGSK

SEQ ID NO: 31 - protein, 11 aa, Artificial Sequence
  Synthetic peptide
TFKDEAGGGSK

SEQ ID NO: 32 - protein, 11 aa, Artificial Sequence
  Synthetic peptide
AFKDEIGGGSK

SEQ ID NO: 33 - protein, 11 aa, Artificial Sequence
  Synthetic peptide
TAKDEIGGGSK

SEQ ID NO: 34 - protein, 11 aa, Artificial Sequence
  Synthetic peptide
TFADEIGGGSK

SEQ ID NO: 35 - protein, 11 aa, Artificial Sequence
  Synthetic peptide
TFKAEIGGGSK

SEQ ID NO: 36 - protein, 11 aa, Artificial Sequence
  Synthetic peptide
THKDEIGGGSK

SEQ ID NO: 37 - protein, 11 aa, Artificial Sequence
  Synthetic peptide
TFQDEIGGGSK

SEQ ID NO: 38 - protein, 10 aa, Artificial Sequence
  Synthetic peptide
THQDEIYEQK

SEQ ID NO: 39 - protein, 28 aa, Artificial Sequence
  Synthetic peptide
PNIAEQYTHQDEIYEQVHSKGLYVPESR

SEQ ID NO: 40 - protein, 9 aa, Artificial Sequence
  Synthetic peptide
THQDEIYEQ

SEQ ID NO: 41 - protein, 10 aa, Artificial Sequence
  Synthetic peptide
THQDEIFEQK